(19) United States
(12) Patent Application Publication
(10) Pub. No.: US 2012/0029266 A1
(43) Pub. Date: Feb. 2, 2012

(54) ANISOTROPIC REINFORCEMENT AND RELATED METHOD THEREOF

(75) Inventors: Jeffrey W. Holmes, Charlottesville, VA (US); Gorav Ailawadi, Charlottesville, VA (US); Gregory M. Fomovsky, Port Washington, NY (US)

(73) Assignee: UNIVERSITY OF VIRGINIA PATENT FOUNDATION, Charlottesville, VA (US)

(21) Appl. No.: 13/263,170

(22) PCT Filed: Apr. 2, 2010
(86) PCT No.: PCT/US10/29813

§ 371 Date: Oct. 14, 2011

Related U.S. Application Data
(60) Provisional application No. 61/166,790, filed on Apr. 6, 2009.

(51) Int. Cl. A61F 2/02

(52) U.S. Cl. 600/16

(57) ABSTRACT

Anisotropic reinforcements and synthetic materials are provided in which the fibers, mesh, weave, or otherwise interlaced or networked components thereof are oriented in one direction so as to create greater stiffness in the one direction of the patch relative to other directions of the reinforcement. Methods of producing such anisotropic reinforcements are provided. The anisotropic reinforcements are advantageously suitable for the surgical repair of incisions, openings, defects, etc. of the cardiovascular system and allow healing to occur while preserving mechanical function, particularly ventricular function.

### CROSS REFERENCE TO RELATED APPLICATIONS

The present application claims priority from U.S. Provisional Application Ser. No. 61/166,790 filed Apr. 6, 2009, entitled “Anisotropic Reinforcement of Myocardial Scar Tissue and Related Method;” the disclosure of which is hereby incorporated by reference herein in its entirety.

### FIELD OF THE INVENTION

The present invention relates generally to improved patches and synthetic materials for use in the repair of body or muscle tissue, body or muscle wall, and vessel defects, particularly in the surgical repair of cardiovascular problems associated with the mammalian heart, blood vessels and aortic vessels.

### BACKGROUND OF THE INVENTION

Each year, nearly 600,000 Americans experience a new heart attack (myocardial infarction); of these, 75% of men and 62% of women survive for at least one year. In addition, each year nearly 300,000 Americans experience a recurrent infarction. As a result, a large portion of the practice of clinical cardiology is currently devoted to management of patients with a heating or healed myocardial infarct.

Unlike many other tissues in the body, heart muscle (myocardium) cannot regenerate. Once myocardium dies during a heart attack, it is gradually n replaced by scar tissue over the course of several weeks. Although the mechanical properties of healing myocardial infarcts are a critical determinant of both depression of pump function and the transition to heart failure, no currently approved drug, method, medium or device is based on the idea of altering infarct mechanical properties as is accomplished by the various aspects of embodiment of the present invention.

Defects, openings, or wounds in the body wall, such as smooth muscle wall, frequently cannot be closed after surgery with autologous tissue due to necrosis, trauma, or other causes. perpendicular to the slits, cuts, or openings in the reinforcement material relative to a reinforcement in the absence of the slits, etc.

In an aspect of an embodiment, anisotropic reinforcements produced by the methods disclosed herein are provided.

In another aspect, an improved implantable anisotropic reinforcement is provided for use in the surgical repair, amelioration, or restoration of body tissue, body walls, muscle walls and vessels. According to this aspect, the anisotropic reinforcements are particularly suited for the surgical repair and restoration of the cardiovascular system, e.g., myocardium, blood vessels, and aortic vessels. The anisotropic reinforcement is suitable for use following cardiovascular surgery to repair a muscle wall defect, such as a heart defect, opening, infarct, wound, etc., or in the repair of blood or aortic vessels in mammals. In accordance with this embodiment, the reinforcement material is stiffer in one single direction than in other directions, thereby creating anisotropy that advantageously mimics the structure observed in scar tissue of some mammalian hearts undergoing healing.

In an aspect of an embodiment, anisotropic reinforcements and synthetic materials are provided for use in methods of repairing, restoring, or ameliorating a lumen comprising anatomical vessels or passageway's of the body, for example, a duct, the lumen of the gut, blood vessels, arteries and aortic vessels. In accordance with various embodiments, the reinforcements can be used as material for insertion with a stent into a vessel, duct, or lumen, for example. For application in lumen repair, e.g., large arteries, the stiffer direction of the anisotropic reinforcements and synthetic materials of various embodiments disclosed herein can advantageously be oriented around the circumference of the vessel, for example, during a surgical procedure in which the reinforcement or synthetic material is used.

An aspect of an embodiment provides a method of repairing, reinforcing, or ameliorating an opening, defect, wound, incision, and the like, in (i) a body or muscle wall; (ii) the cardiovascular system; (iii) the myocardium; (iv) a body vessel or duct, e.g., a blood vessel, an artery, an aortic vessel, or intestinal or bile duct, which involves implanting an anisotropic reinforcement as described herein over the opening, defect, wound, incision, and the like.

An aspect of an embodiment provides a method of strengthening a weakness in a body or muscle wall, such as a hernia, which involves applying an anisotropic reinforcement as made or described herein in the area of the body or muscle wail weakness so as to strengthen it. In another aspect, this embodiment provides a method of strengthening a weakness in myocardial tissue, e.g., the heart, which involves applying an anisotropic reinforcement as made or described herein in the area of the myocardial tissue weakness so as to strengthen it. In another aspect, this embodiment provides a method of strengthening a weakness in a vessel or passageway of the body, such as a genitourinary vessel or duct, a gastrointestinal vessel or duct, a blood vessel, an artery, or an aortic vessel, etc., which involves applying an anisotropic reinforcement as made or described herein in the area of vessel weakness so as to strengthen it.

Additional aspects, features and advantages afforded by the various embodiments will be apparent from the detailed description and exemplification herein.

Unlike conventional approaches, an aspect of various embodiments provides the ability to intentionally create anisotropy for cardiac applications to improve heart function.

Unlike conventional approaches, an aspect of various embodiments provides a product, composition and method that is designed to improve heart function in patients who have had a heart attack, but are not yet in heart failure. Accordingly, an aspect of various embodiments offers an entirely new market: any patient who has had a heart attack, but has not yet progressed to heart failure.

An aspect of an embodiment of the present invention provides a reinforcement for communication with the heart. The reinforcement may be configured to create stiffness in one direction relative to other directions of the reinforcement, thereby reinforcing a region of the heart for improving heart function. It should be appreciated that the configuration may be accomplished by 1) an attachment technique (i.e., process or method) itself, 2) the existing configuration of the reinforcement as provided prior to the attaching, or 3) a combination of the attaching technique as well as the existing structure or material of the reinforcement. principles of the invention. The drawings are provided only for the purpose of illustrating select embodiments of the invention and are not to be construed as limiting the invention.

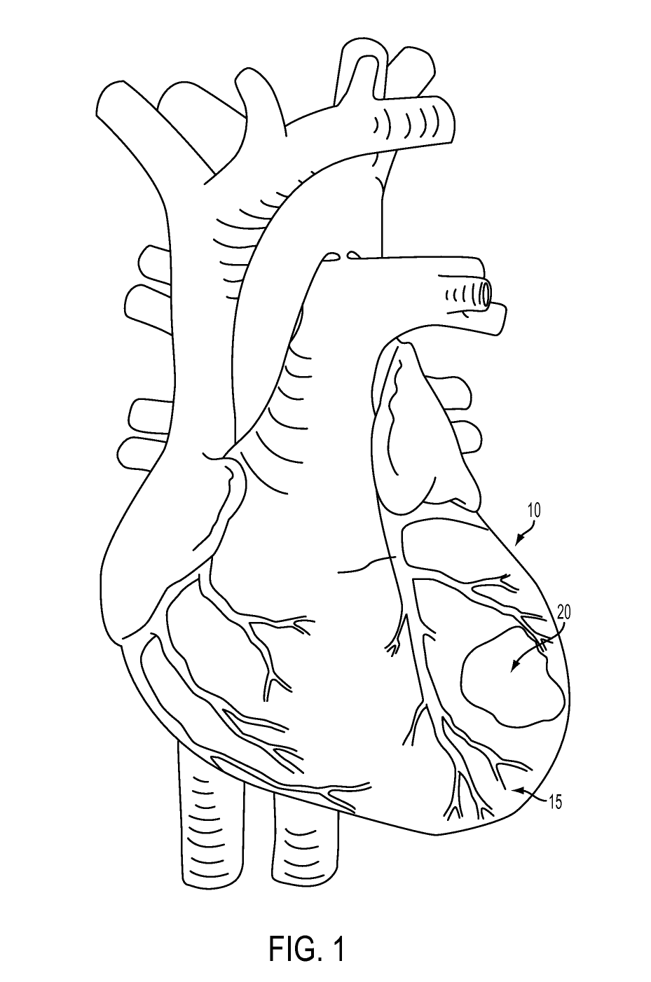

FIG. 1 schematically illustrates a reinforcement in communication with the heart.

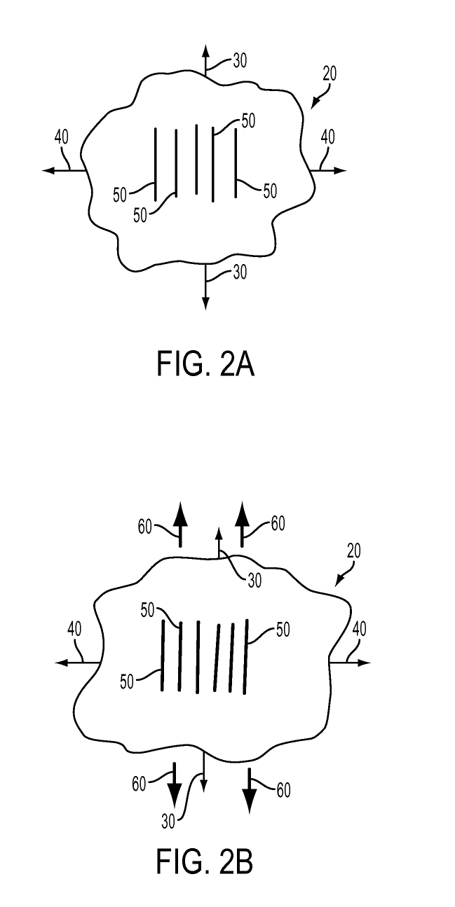

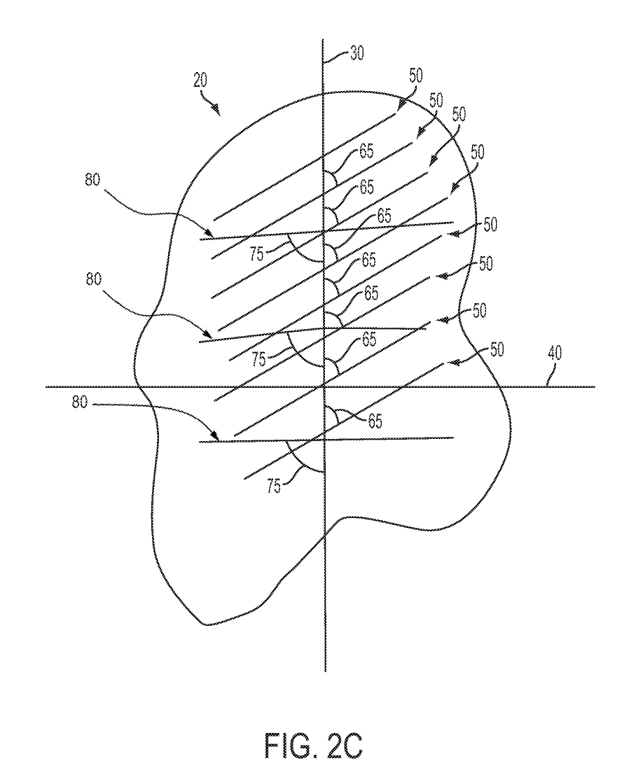

FIG. 2A schematically illustrates the reinforcement.

FIG. 2B schematically illustrates the reinforcement; and illustrates the longitudinal stiffness that it provides.

FIG. 2C graphically illustrates the reinforcement having fibers of various alignment angles.

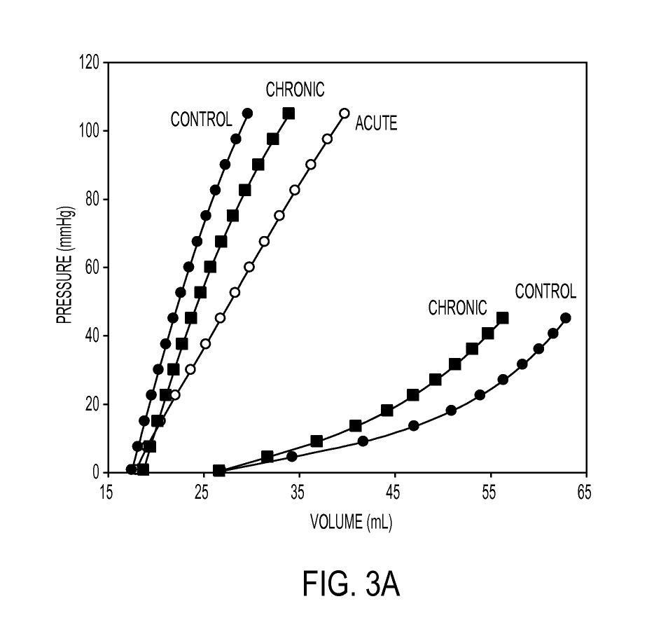

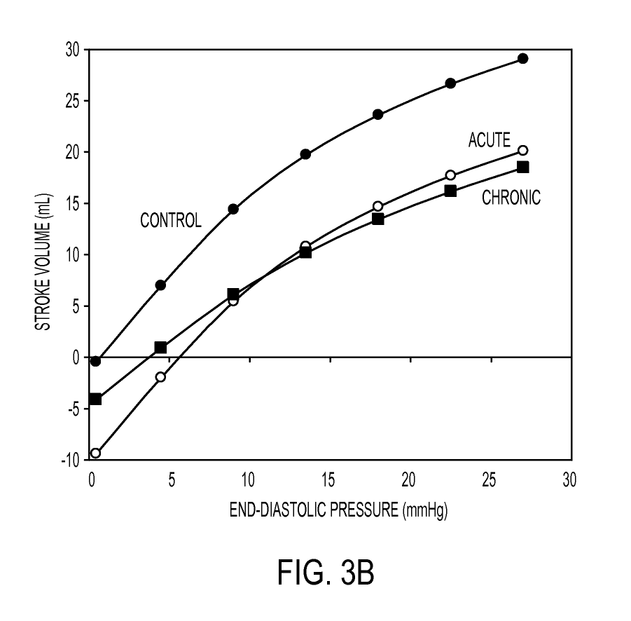

FIG. 3A graphically illustrates through pressure (mmHg) vs. Volume (mL) the net amount of blood the heart pumps at a particular filling pressure decreases immediately following infarction (“acute”) due to depressed systolic function, and may not substantially change when the scar is isotropically stiff (“chronic”), because improvements in systolic function are offset by increased diastolic stiffness.

FIG. 3B graphically illustrates the net amount of blood the heart pumps at a particular filling pressure is depressed by myocardial infarction (“acute”) and may not substantially change when the scar is isotropically stiff (“chronic”) through stroke volume (mmHg) vs. end-diastolic pressure axes (mL).

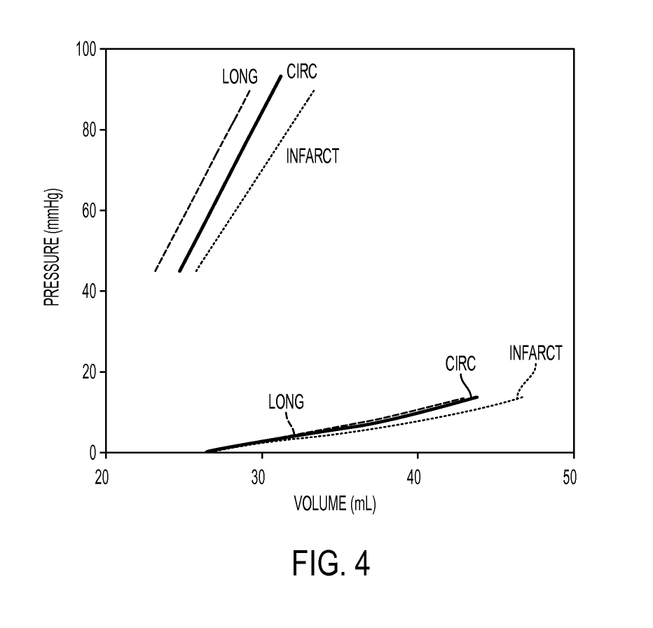

FIG. 4 as graphically illustrated, computer simulations of a large antero-apical infarct suggest that longitudinal reinforcement (“long”) improves systolic function more that circumferential reinforcement (“circ”), with similar effects on diastolic function.

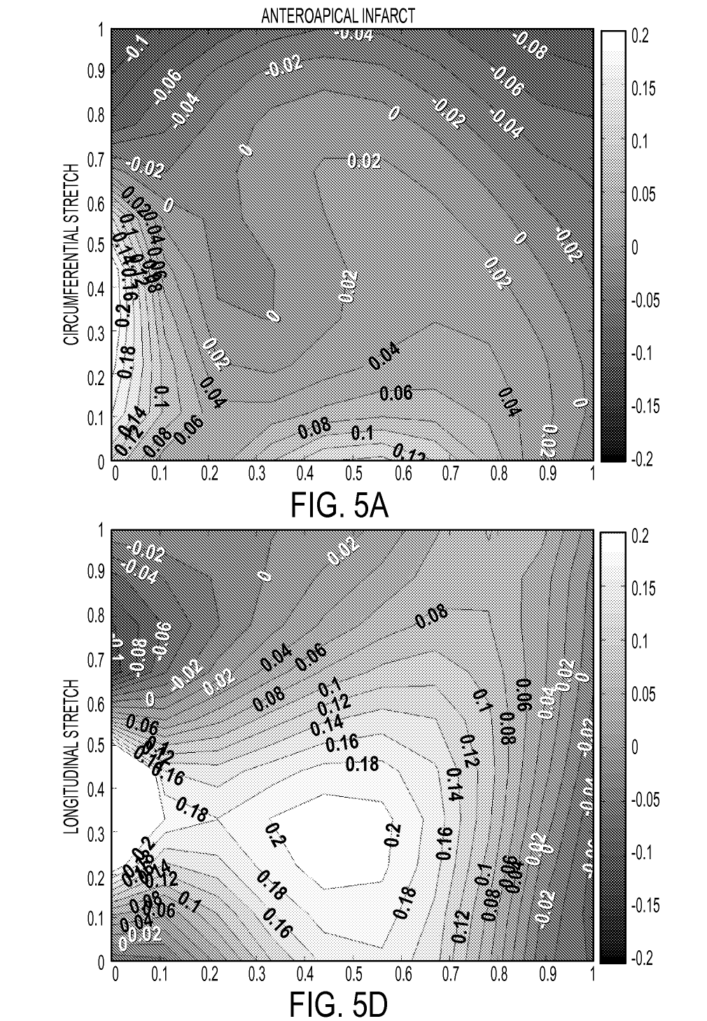

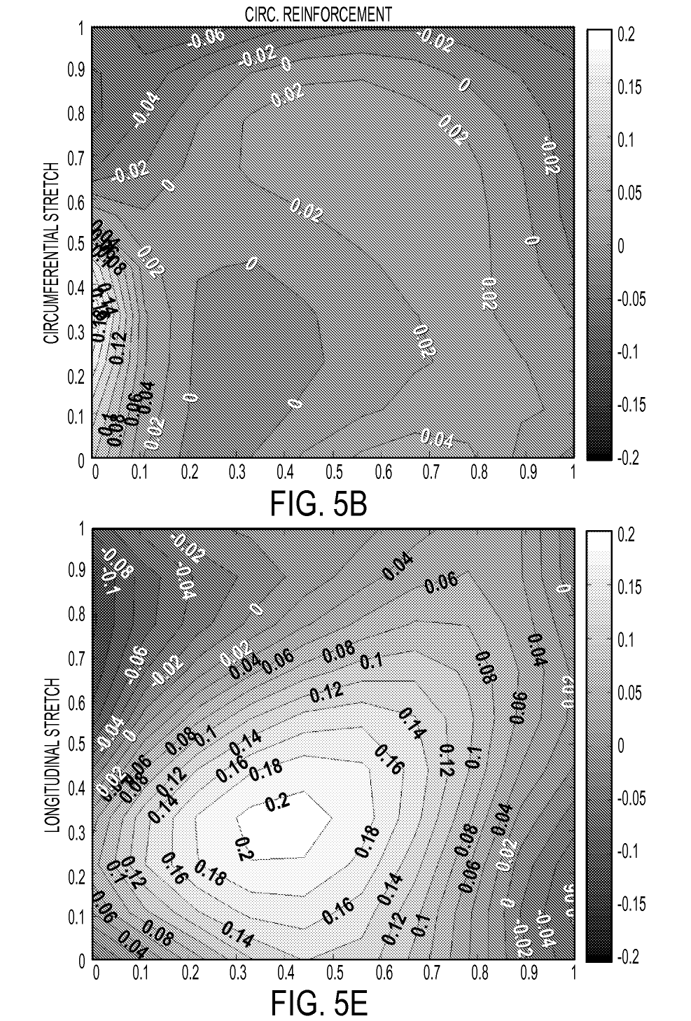

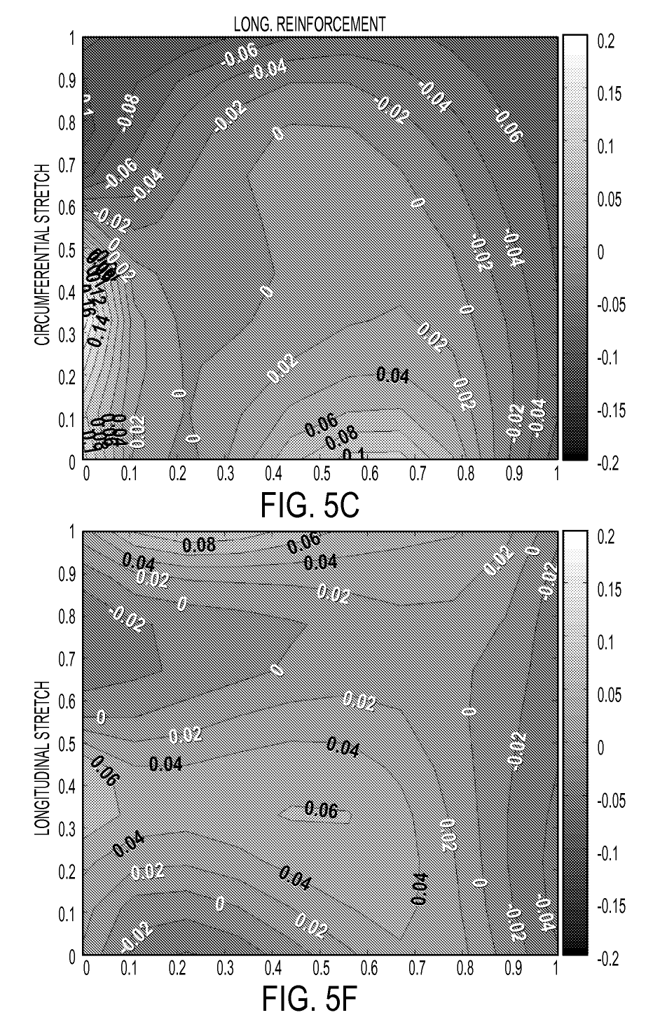

FIGS. 5A-5F graphically illustrate the large antero-apical infarcts may stretch significantly in the longitudinal direction, but not much in the circumferential direction. circumferentially around the heart, so as to preserve ventricular and overall function of the heart during the course of post-infarction healing.

More specifically, and without wishing to be bound by theory, scar anisotropy during cardiac heating permits the scar to resist circumferential stretching while allowing the scar to deform normally and compatibly with non-infarcted tissue in the longitudinal and radial directions. It should be appreciated that the presence of an infarct may interfere with the pumping function of the ventricle by reducing the proportion of the ventricular wall that contributes to blood ejection. Also, an infarct may locally stretch during systole, thereby absorbing part of the energy generated by the ventricle and reducing ejection work. Thus, both progressive shrinkage and stiffening of the healing infarct would be expected to improve ventricular function. According to an aspect of an embodiment, an anisotropic reinforcement or synthetic material that is stiffer, or more rigid and taut, in one direction provides the ability to resist stretch in some directions and the freedom to deform with the surrounding myocardium in other directions.

An aspect of an embodiment may provide the ability to selectively reinforce scar tissue in one direction to create anisotropy in scars that did not normally possess it and may improve heart function and pump function in the heart after a myocardial infarction. One aspect of an embodiment may comprise selectively reinforcing myocardial scar tissue in one direction to improve heart function and pump function. This may require at least a determination of which direction to reinforce the scar (which may be different for different scars), as well as selective reinforcing the scar in that direction.

In accordance with an embodiment, a non-limiting, exemplary method for determining the direction to reinforce the scar may be to image the scar during contraction of the heart, and reinforce the scar in the direction of greatest stretching.

In accordance with an embodiment, some methods of reinforcing a scar may include modifying a stiff biocompatible material appropriate for cardiovascular surgeries (e.g. Dacron patches currently used to repair ventricular aneurysms) to render the scar stiff in only one direction, and sewing the material to the epicardial surface of the heart. One method of modifying the material may be to cut substantially parallel slits or elongated apertures in the material, which may render it more deformable in the perpendicular direction to the slits than parallel to them. Another method for selectively reinforcing a scar may be to create new biocompatible fabrics using weaving patterns customized to provide the desired level of anisotropy, and sew them to the epicardial surface of the heart. Another method for selectively reinforcing a scar may be to attach the ends of strips of a stiff material such as existing cardiovascular fabrics to the epidcardial surface of the heart so that the long axis of the strips is oriented substantially in the desired direction of reinforcement. The desired direction may be, for example, but not limited thereto: a substantially longitudinal or circumferential direction of the heart; substantially aligned with (i.e. parallel to) or transverse to the underlying muscle fiber direction of the heart and/or collagen fiber direction of said infarct region. Another method for selectively reinforcing a scar may be to modify an existing soft biocompatible material to make it stiff in substantially only one direction, and sew the customized material to the epicardial surface of the heart. A nonlimiting example of such a modification may be to reinforce the outer surface of a soft biocompatible material such as silicone with a stiff biocompatible material such as nitinol wire. Another method for selectively reinforcing a scar may be to create new composite materials having different components, such as providing stiffness in one direction and providing flexibility in a substantially perpendicular direction. Another method for selectively reinforcing a scar may be to chemically treat a fibrous biocompatible material to render it anisotropic.

While the aforementioned methods may involve sewing something to the epicardial surface, they may comprise other attachment means as well, such as adhesion. An aspect of an embodiment may be the attachment provided in patients who are already undergoing open-heart coronary bypass surgery after a heart attack. Additionally, the reinforcement of a scar may be performed using minimally invasive approaches, which may widen the appropriate commercial market to any patient who has scar tissue from a prior heart attack. Additionally, while the epicardial surface is used in an exemplary fashion, all of the methods listed could also be used to reinforce the inner surface of the heart.

During normal healing, scar tissue may become stiffer. Prior theory assumed that scars were stiff in all directions (isotropic). An aspect of various embodiments includes unexpected and surprising results. The results disclosed herein indicate that while some scars are isotropic, others may in fact he anisotropic. Immediately after a heart attack, systolic function may be depressed because the soft damaged region bulges instead of contraction when the heart generates pressure with each beat. The diastolic relationship may remain unchanged. An isotropically still infarct bulges less, improves systolic function, but the increased stiff may impair diastolic function (filling). The balance between these two effects may best be illustrated with a cardiac output curve, as shown in FIG. 3A, which illustrates through pressure (mmHg) vs. Volume (mL) axes that the net amount of blood the heart pumps at a particular filling pressure may not substantially change when the scar is isotropically stiff. Similarly, FIG. 3B illustrates the net amount of blood the heart pumps at a particular filling pressure may not substantially change when the scar is isotropically stiff through stroke volume (mmHg) vs. end-diastolic pressure axes (mL).

Circumferential stiffening or reinforcement may have a similar effect to isotropic stiffening—systolic function may improve, but some diastolic function may be lost. Longitudinal stiffening, however, further improves systolic function without additional effects on diastolic function. FIG. 4 illustrates the pressure (mmHg)-volume (mL) relationships predicted by computer simulations. FIG. 4 graphically illustrates the effect of stiffening the infarct in just one direction. The acute infarct is identified as “infarct” on the graph.” Circumferential stiffening or reinforcement has a similar effect to isotropic stiffening—systolic function improves, but sonic diastolic function is lost. The circumferential stiffening is identified as “circ” on the graph. Longitudinal stiffening further improves systolic function without additional effects on diastolic function. Overall, longitudinal stiffening improves both stroke volume (volume pumped per beat) and ejection fraction more than circumferential reinforcement. The longitudinal stiffening is identified as “long” on the graph.

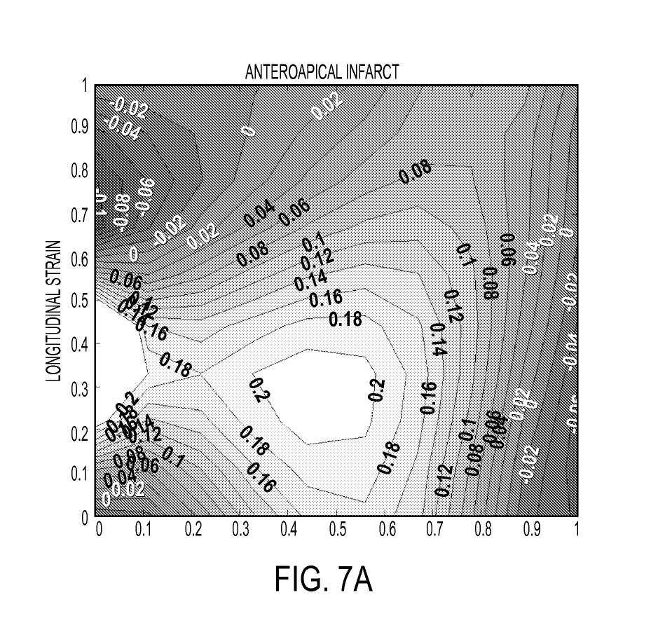

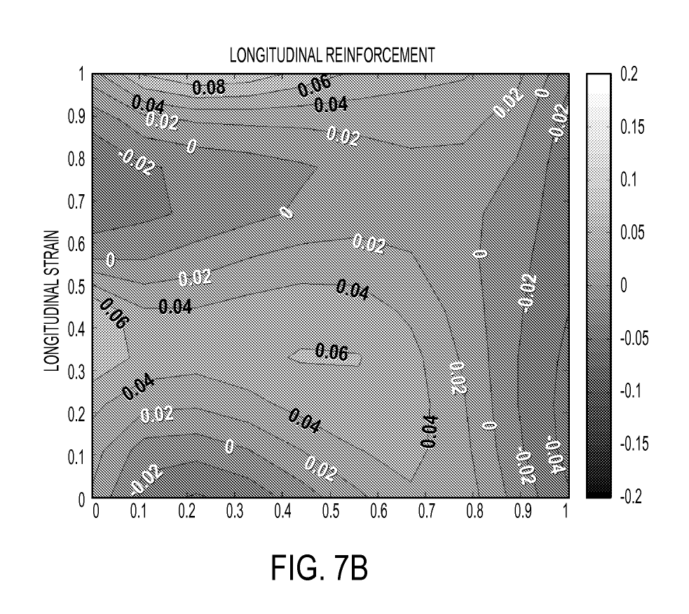

It should be noted that FIG. 5D and FIG. 5F are enlargements of FIG. 7A and FIG. 7B, respectively. FIG. 5 illustrates the underlying reason that the counter-intuitive longitudinal reinforcement is effective, while intuitive circumferential reinforcement is not effective. The white areas of the illustration are circumferential and longitudinal stretching, and the dark areas are circumferential and longitudinal shortening in an antero-apical infarct region during contraction of the heart. Large antero-apical infarcts may stretch significantly in the longitudinal direction, but not much in the circumferential direction (FIGS. 5A and 5D). For this reason, reinforcing in the circumferential direction did not provide much effect (FIGS. 5B and 5E), but longitudinal reinforcement had a substantial effect (FIGS. 5C and 5F).

In accordance with an aspect of an embodiment, it should be noted that the pattern of stretch in an infarction may be different in infarcts in different locations of the heart. While the exact ratio of stiffness in the longitudinal and circumferential directions may not be absolutely critical, as long as one direction is substantially stiffer, such as 20 to 40 times stiffer, choosing the proper orientation for the stiffer direction may be critical.

In accordance with an aspect of an embodiment, FIG. 1 illustrates a reinforcement 20 for communication with the heart 10. For illustration purposes, and not intended to be limiting in any aspect, the reinforcement is shown at the Left Ventricle 15 The reinforcement may be configured to create stiffness in one direction relative to other directions of the reinforcement for reinforcing a region of the heart for improving heart function. This configuration provides for anisotropic reinforcement. The configuration may be achieved by an attachment technique of the reinforcement to the heart. The configuration may be provided whereby the anisotropic reinforcement configuration prior to the attachment to the heart. The configuration may also be provided by an attachment technique to the heart. Also, the anisotropic properties may be provided by both the design of the reinforcement combined with the attachment technique.

In accordance with an aspect of an embodiment, the heart function improved by the anisotropic reinforcement may comprise pump function. Additionally, the heart function may comprise at least one of cardiac output, ejection fraction, volumes, stroke volume, pressures, end-diastolic volume (EDV), end-systolic volume (ESV), energetics, energetic efficiency, and need for inotropic support or the like. The region of the heart reinforced may comprise at least one of, at least a portion of a wall, ischemic, infarct, epicardial surface, or inner surface.

In accordance with an aspect of an embodiment, the communication of the reinforcement to the heart may comprise at least one of adhesion, attachment, or suture. Additionally, the anisotropic reinforcement may comprise at least one of a graft, patch, member, local-reinforcement, substrate, material, wire, reinforcing member, members applied to the heart, members into the heart, support, brace, buttress, coating, augmentation, or fortification. The anisotropic reinforcement may further comprise a patch with at least substantially parallel slits cut into said patch to decrease stiffness of the reinforcement in the direction at least substantially perpendicular to the slits. Additionally, the reinforcement may provide flexibility in the direction at least substantially perpendicular to the stiffness.

In accordance with an embodiment, the anisotropic reinforcement may comprise fibers oriented in one direction of the reinforcement to create stiffness in the one direction relative to other directions of the reinforcement. The fibers oriented in the one direction of the reinforcement may comprise a plurality of fibers relative to the fibers in the other directions of the reinforcement. The fibers in the one direction of the reinforcement may be oriented in at least a substantially straight line relative to randomly or stochastically placed fibers in other directions of the reinforcement. The fibers in the one direction of the reinforcement may be tight, or less slack relative to fibers in other directions of the reinforcement. Pores or apertures within the fibers in the one direction of the reinforcement may be closer in proximity to each other than pores or apertures within the fibers in other directions of the reinforcement. The fibers oriented in the one direction of the reinforcement may be denser relative to the fibers in other directions of the reinforcement. The fibers oriented in the one direction of the reinforcement may be reinforced in the one direction relative to the fibers in other directions of the reinforcement. In this case, the fiber reinforcement may comprise at least one of: additional fibers, natural fibers, synthetic fibers, mesh, collagen fibers, metals, cloth, or biocompatible metals. In the case of biocompatible metals, the biocompatible metals may be selected from stainless steel, titanium, metal alloys, or a combination thereof. In the case of metal alloys, the metal alloys may be selected from: In—Ti, Fe—Mn, Ni—Ti, Ag—Cd, Au—Cd, Au—Cu, Cu—Al—Ni, Cu—Au—Zn, Cu—Zn, Cu—Zn—Al, Cu—Zn—Sn, Cu—Zn—Xe, Fe₃Be, Fe₃Pt, Ni—Ti—V, Fe—Ni—Ti—Co, or Cu—Sn.

In accordance with an embodiment the anisotropic reinforcement may comprise a synthetic material. In this case, the synthetic material may be selected from tantalum gauze, stainless steel mesh, DACRON, ORLON, FORTISAN, nylon, knitted polypropylene (MARLEX), microporous expanded-polytetrafluoroethylene (GORE-TEX), Dacron-reinforced silicone rubber (SILASTIC), polyglactin 910 (VICRYL), polyester (MERSILENE), polyglycolic acid (DEXON), or a combination thereof.

As illustrated in FIGS. 2A and 2B, the reinforcement 20 may comprise fibers 50 or the like that are aligned longitudinally in a single direction (or at least substantially single as required) in the reinforcement 20 to result in increased stiffness in the longitudinal direction 60, relative to the reinforcement's circumferential axis 40. The fibers may be aligned in the single direction (or at least substantially single as required) along the reinforcement's longitudinal axis 30.

In accordance with an embodiment, the anisotropic reinforcement may comprise fibers aligned in a single direction for increased stiffness of the reinforcement in the direction of fiber alignment relative to directions of fiber nonalignment. The fibers may be aligned longitudinally in the single direction in said reinforcement to result in increased stiffness in the longitudinal direction. The fibers may be aligned in the single direction along the reinforcement's longitudinal axis. Additionally, the fibers aligned in the single direction may be a larger size relative to the size of the fibers in other directions of the reinforcement. The fibers aligned in the single direction may be reinforced in the single direction relative to the fibers in other directions of the patch.

In accordance with an embodiment, the anisotropic reinforcement may comprise interwoven fibers, wherein a plurality of fibers may be oriented at least substantially in a single direction within the reinforcement to produce increased stiffness in the single direction relative to other directions. The other directions may include at least substantially perpendicular or diagonal thereto. Additionally, the plurality of fibers may be oriented in the longitudinal direction of the reinforcement relative to fibers in the substantially circumferential, radial, perpendicular, or diagonal directions of the reinforcement. The plurality of fibers may also be the same number and/or material as the fibers comprising the reinforcement, or may be different in number and/or material from the fibers comprising the reinforcement.

In accordance with an embodiment, the anisotropic reinforcement may comprise strips of a stiff material attached to the region of the heart such that the longitudinal axis of the strips may be oriented in a desired direction of reinforcement. The strips may be integrally connected and/or separate from one another. Additionally, the stiff material may comprise cardiovascular fabrics.

In accordance with an embodiment, the anisotropic reinforcement may comprise a region located in one area of the reinforcement to create stiffness in the one area relative to other regions of the reinforcement. The region may comprise a plurality of fibers relative to the other regions of the reinforcement. The region may be oriented in at least a substantially straight line relative to other regions of reinforcement. The region may be tight, or have less slack relative to other regions of reinforcement. Additionally, the region may be denser relative to other regions of the reinforcement.

In accordance with an embodiment, the region may also be further reinforced relative to other regions of the reinforcement. The region of further reinforcement may comprise at least one of: fibers, additional fibers, natural fibers, synthetic fibers, mesh, collagen fibers, metals, cloth, or biocompatible metals. In the case of biocompatible metals, the biocompatible metals may be selected from stainless steel, titanium, metal alloys, or a combination thereof. In the case of metal alloys, the metal alloys may be selected from: In—Ti, Fe—Mn, Ni—Ti, Ag—Cd, Au—Cd, Au—Cu, Cu—Al—Ni, Cu—Au—Zn, Cu—Zn, Cu—Zn—Al, Cu—Zn—Sn, Cu—Zn—Xe, Fe₃Be, Fe₃Pt, Ni—Ti—V, Fe—Ni—Ti—Co, or Cu—Sn. Additionally, the region may be aligned longitudinally in a single direction in said reinforcement to result in increased stiffness in the longitudinal direction of the reinforcement.

In accordance with an embodiment, the region may be aligned in a single direction along the reinforcement's longitudinal axis, relative to the reinforcement's other regions. The other regions may include at least substantially perpendicular or diagonal regions of the reinforcement. The region may be oriented in the longitudinal direction of the reinforcement relative to regions in the substantially circumferential, radial, perpendicular, or diagonal directions of the reinforcement. The reinforcement may provide flexibility in a direction at least substantially perpendicular to the stiffness. The reinforcement may provide flexibility in a direction at least substantially perpendicular to the stiffness.

In accordance with an embodiment, the anisotropic reinforcement may be chemically treated to create anisotropy. Additionally, the anisotropic reinforcement may be mechanically treated to create anisotropy. In the case of mechanical treatment, the mechanical treatment may comprise at least one of grinding, finishing, abrading, inflating, shrinking, directionally-specific shrinking, inducing tension, slacking, coating, or expanding. The reinforcement may also comprise shape memory material or structure, pre-stressed material or structure, recoil material or structure, active recoil material or structure, or pre-shaped material or structure, as well as any combination thereof. An example of shape memory material includes, but not limited thereto, nitinol or the like. In an embodiment, the reinforcement (or portions or regions thereof) may be designed to be elastic. For instance, the reinforcement (or portions or regions thereof) has the capability to recoil in the one appropriate direction (or directions). For example, an aspect may provide a reinforcement in a direction that has recoil properties. For example, an aspect may provide a reinforcement that may actively recoil.

In accordance with an aspect of an embodiment, the anisotropic reinforcement may be configured to provide a method and design for improving heart function. For instance, the method includes determining the direction to reinforce an infarction and configuring it accordingly. The reinforcement is configured for selectively reinforcing the infarction. The process of selectively reinforcing provides, but not limited thereto, an anisotropic reinforcement. Some exemplary ways of determining such direction(s), etc. include, but not limited thereto, a clinical assessment or medical practitioner assessment of the infarction. In addition or conjunction therewith, the determination may be provided by imaging the infarction.

In an embodiment, the configuration to provide the reinforcement may be accomplished by 1) providing a reinforcement that already possesses anisotropic properties and combining it with 2) a method or process of communicating or disposing the reinforcement (or portions thereof, as well as additional portions or material(s)) to or with the heart so as to further provide additional anisotropic properties as required or desired.

Alternatively, in an embodiment, the configuration to provide the reinforcement may be accomplished solely by a method or process of communicating or disposing a reinforcement (or portions thereof) or material(s) to or with the heart.

It should be appreciated that the method or process of communicating or disposing the reinforcement or material(s) to or with the heart may include, but not limited thereto, adhering, attaching, and suturing said reinforcement with said heart.

An aspect of an embodiment provides a method or process of reinforcing a heart possessing an infarction, whereby the reinforcing creates a reinforcement to provide stiffness in one direction relative to other directions of the reinforcement. In turn, this creation preferentially reinforces one direction of the infarct region of the heart wall. In an embodiment, the preferential reinforcement provides the stiffness in at least one direction of the reinforcement that is at least substantially aligned with the underlying muscle fiber direction of the heart and/or collagen fiber direction of the underlying infarct region. In an embodiment, the preferential reinforcement provides the stiffness in at least one direction of the reinforcement that is at least substantially transverse with the underlying muscle fiber direction of the heart and/or collagen fiber direction of the underlying infarct region. It should be appreciated that the reinforcing improves heart function, as well as may provide other functions, mechanical integrity and operation.

In accordance with an embodiment, fibers may be oriented in one direction of said reinforcement and may be distributed over a smaller range of angles to produce stiffness in a direction, relative to other directions having fibers distributed over a larger range of angles. As shown in FIG. 2C, the fibers 50, 80 have various alignment angles 65, 75, respectively. The reinforcement may comprise smaller angles of fibers comprising one direction of the reinforcement to produce stiffness in the one direction relative to larger angles of the fibers comprising other directions of the reinforcement. The fibers 50 with sharper angles of alignment 65 is contrasted with fibers 80 with larger angles of alignment 75. The angle of alignment may vary as required. For example, the stiffness in the one direction of the reinforcement may comprise fibers oriented having the alignment angles within about 10 degrees to less than about 90 degrees relative to the local circumferential axis of the reinforcement 40. The stiffness in the one direction of the reinforcement may comprise fibers oriented having the alignment angles within about 20 degrees to about 70 degrees relative to the local circumferential axis 40 of the reinforcement. The stiffness in the one direction of the reinforcement may comprise fibers oriented having the alignment angles within about 25 degrees to about 50 degrees relative to the local circumferential axis of the reinforcement. The stiffness in the one direction of the reinforcement may comprise fibers oriented having the alignment angles within about 30 degrees to about 45 degrees relative to the local circumferential axis of the reinforcement.

In accordance with an embodiment, the anisotropic reinforcement may be configured to provide at least one of: a drug treatment, cellular therapy, pacing capabilities, stem cell therapy, or mechanical integrity.

In accordance with an embodiment, an anisotropic reinforcement may be provided for communication with a heart possessing an infarction, whereby said reinforcement may be configured to create stiffness in one direction relative to other directions of said reinforcement, to preferentially reinforce one direction of the infarct region of the heart wall. The preferential reinforcement may provide said stiffness in at least one direction of said reinforcement that may be at least substantially aligned with said infarction. The preferential reinforcement may also provide said stiffness in at least one direction of said reinforcement that may be at least substantially transverse with said infarction.

In accordance with an embodiment, the three dimensional orientation of the anisotropic reinforcements on the heart can be similar to the orientation of scar tissue fibers that occur in normal heart tissue. Such fibers are oriented circumferentially around the heart. Accordingly and without limitation, alignment of the stiffer direction of the reinforcement material of an embodiment is with the circumference of the heart so as to maintain similarity to scar tissue fiber orientation. In addition, for lumen and vessel repair, the stiffer direction of the reinforcement material of an embodiment can be oriented around the circumference of the lumen or artery during surgical implantation. When the anisotropic reinforcements and synthetic materials of various embodiments are employed to repair other mechanically anisotropic tissues, such as skin, muscle, tendon, gut, etc., the stiffer direction of the anisotropic reinforcement material can be advantageously aligned with the axis of greatest stiffness of the neighboring normal tissue.

The unique anisotropic reinforcements of various embodiments are comprised of fibers, threads, weave, mesh, or otherwise interlaced or networked components, that are oriented in one predominant direction relative to the fibers, threads, weave, mesh, or otherwise interlaced or networked components in other directions of the reinforcement that are not directionally oriented. In this manner, the reinforcements of an embodiment provide mechanical properties akin to the anisotropic collagen fiber orientation in actual scar tissue following cardiac defect repair or post-infarction healing. It is an advantage that the reinforcements of various embodiments are anisotropic and do not have the same stiffness in all directions, because they can better preserve the overall functioning of a repaired heart or vessel by better replacing the mechanical function of the repaired region and by improved compatibility with adjacent anisotropic tissue.

The anisotropic reinforcements of an embodiment are suitable for use in the repair of a variety of heart and vessel defects, disorders, dysfunctions, abnormalities, openings, incisions, wounds and the like. Such reinforcements can be used in the repair of congenital heart defects as well as defects and infarctions in older patients. As an nonlimiting example, one in about 1500 babies is born with an atrial septal defect (ASD), which is a hole in the heart chamber. Open-heart surgery during childhood is the conventional form of treatment. One alternative treatment for nearly 50-60% of cases involves the use of an experimental procedure known as “Helex”, which can be accomplished via catheterization through a leg vein, rather than open-heart surgery. The Helex system was created to close holes in the heart in cases of ASD or ventricular septal defects and is based on technology that uses two discs, one to cover the hole from the left side of the heart and one to cover the hole from the right side of the heart. These two discs stick together to form a patch. The Helex device includes a wire frame made of nickel titanium metal, while the reinforcement covering is made out of a type of GORE-TEX®, which will last for a lifetime. Such reinforcements can be created to be anisotropic according to the various embodiments disclosed herein.

Advantageously, commercially-available, synthetic, isotropic patch materials are suitable for use as starting materials to produce the anisotropic reinforcements in accordance with the methods of various embodiments. In addition, the anisotropic reinforcements of select embodiments can be newly engineered, e.g., using materials that are similar or identical to materials that are used to make commercially-available patches. Illustratively, several types of suitable synthetic materials that have been used in body or muscle wall or vessel repair are useful in an embodiment disclosed herein and include, without limitation, tantalum gauze, stainless steel mesh, DACRON®, ORLON®, FORTISAN®, nylon, knitted polypropylene (MARLEX®), microporous expanded-polytetrafluoroethylene (GORE-TEX®), dacron-reinforced silicone rubber (SILASTIC®), polyglactin 910 (VICRYL®), polyester (MERSILENE®), polyglycolic acid (DEXON®) or a combination thereof. Other materials that can be used with various embodiments are processed sheep dermal collagen (PSDC®), crosslinked bovine pericardium (PERI-GUARD®) and preserved human dura (LYODURA®), or any combination thereof.

An aspect of an embodiment provides synthetic meshes comprising woven fibers that are advantageously easily fabricated and are malleable as desired for preparing the anisotropic reinforcements. Except for nylon, synthetic meshes retain their tensile strength in the body. In addition, metallic meshes are inert, resistant to infection and can stimulate fibroplasia. Other synthetic materials suitable for preparing implantable anisotropic reinforcements and synthetic materials in accordance with various embodiments disclosed herein are also encompassed. Such materials are suitably chemically inert, noncarcinogenic, capable of being fabricated in the form required, capable of resisting mechanical stress, sterilizable, not physically modified by tissue fluids, not prone to exciting an inflammatory or foreign reaction in the body, not prone to inducing an allergic or hypersensitive state, and not prone to promoting visceral adhesions.

The biocompatible synthetic anisotropic reinforcements of an embodiment can be engineered or fabricated to produce an anisotropic product having the mechanical property of being stiffer in one direction relative to other directions of the patch. The anisotropic reinforcements of an embodiment are created so that they comprise component fibers, weave, mesh, or otherwise interlaced or networked components that are oriented or aligned in one predominant direction, while the component fibers, weave, mesh, or otherwise interlaced or networked components in other directions of the reinforcement are not so oriented or aligned. The resulting anisotropic reinforcement does not have the same mechanical properties in all directions, as do currently available synthetic reinforcements and reinforcement materials.

One aspect of an embodiment is illustrated in the following non-limiting way: In general, the reinforcements according to various embodiments can be produced by manipulating the orientation of the fibers of the reinforcement so that the fibers, or additional fibers, for example, are oriented in one direction relative to the fibers in other directions of the patch. An anisotropic reinforcement can comprise more fibers in a single direction compared with other directions of the reinforcement material; for example, by reducing the angles between the fibers as the reinforcement material is rotated to create the reinforcement during production. In addition, a reinforcement can comprise more than one layer of fibers, or more than one layer of fiber-containing material, wherein the reinforcement is made stiffer in one direction relative to other directions. This can be achieved by making the angles of the fibers smaller and smaller as the material is rotated to produce the final reinforcement material. Thus, by way of nonlimiting example, the fiber weave in one direction can be reduced from about 90° in a typical isotropic reinforcement to about 30° in an anisotropic reinforcement to result in the fibers being oriented or aligned in a single direction in the weave of the anisotropic reinforcement relative to other directions to achieve stiffness in the single direction of the patch.

The production of an anisotropic reinforcement in which the fibers are stiffer in one direction relative to other directions can be accomplished in a number of ways. For example and without limitation, the fiber weave of a reinforcement an be engineered to create an anisotropic reinforcement suitable for use in various embodiments by weaving the fibers of the reinforcement to have more slack in one direction versus other directions; weaving the fibers to be straight and thus stiffer in one direction of the patch, while weaving the fibers in other directions to be non-straight, e.g., coiled or randomly woven; weaving the fibers in the reinforcement so that the fiber pore sizes in one direction are smaller than the fiber pore sizes in other directions, resulting in the pores in the one direction in closer proximity to each other than in other directions of the patch; weaving the fibers in one direction of the reinforcement to be tighter or denser than the fibers in other directions; and weaving the fibers in one direction of the reinforcement to be larger in size than are the fibers in other directions of the patch.

in accordance with an embodiment, a method for improving heart function may be provided, comprising: communicating an anisotropic reinforcement with the heart, wherein said reinforcement may be configured to create stiffness in one direction relative to other directions of said reinforcement, for reinforcement of the wall of the heart for said improved pump function.

In accordance with an embodiment, a method for improving heart function may be provided, comprising determining the direction to reinforce an infarction, providing an anisotropic reinforcement with selective reinforcement for said determined direction, and communicating said anisotropic reinforcement with the heart for reinforcing said infarction.

In accordance with an embodiment, determining the direction to reinforce may comprise a clinical assessment of the infarction, or imaging the infarction. In the case of imaging, the imaging may comprise assessment of infarct stretching. This may include the use of MRI, X-Ray, CAT Scan, or Ultrasound technology.

In accordance with an embodiment of providing an anisotropic reinforcement with selective reinforcement may comprise weaving tight fibers in one direction relative to other directions of the anisotropic reinforcement to produce stiffness in the one direction relative to other directions of the anisotropic reinforcement, and may comprise weaving loose fibers in the other directions of the anisotropic reinforcement relative to the one direction. It may also comprise weaving dense fibers in one direction of the anisotropic reinforcement relative to other directions of the anisotropic reinforcement to produce stiffness in the one direction relative to other directions of the anisotropic reinforcement, and may further comprise weaving loose fibers in the other directions of the anisotropic reinforcement relative to the one direction. Providing an anisotropic reinforcement with selective reinforcement may also comprise weaving straight, tight, or stretched fibers in a single direction of the anisotropic reinforcement relative to other directions of the anisotropic reinforcement to produce stiffness in the single direction, relative to other directions of the anisotropic reinforcement. This may further comprise weaving randomly or stochastically oriented fibers in the other directions of the anisotropic reinforcement relative to the one direction, and may further comprise weaving slack or unstretched fibers in the other directions of the anisotropic reinforcement relative to the one direction. In this case, the slack or unstretched fibers may comprise coiled, curved, or zig-zag fibers.

Additionally, providing an anisotropic reinforcement with selective reinforcement may comprise weaving small pore sizes within fibers comprising one direction of the anisotropic reinforcement relative to other directions of the anisotropic reinforcement to create stiffness in the one direction relative to the other directions of the anisotropic reinforcement, and may further comprise weaving larger pore sizes in the other directions of the anisotropic reinforcement relative to the one direction of the anisotropic reinforcement. Providing an anisotropic reinforcement with selective reinforcement may also comprise cutting slits in said anisotropic reinforcement along one direction of said anisotropic reinforcement so that said anisotropic reinforcement stiffens selectively in the direction parallel to the slits. It may also comprise chemically treating said reinforcement to render it anisotropic, such to create stiffness in one direction relative to other directions of the anisotropic reinforcement. It may also comprise mechanically treating said reinforcement to render it anisotropic, such to create stiffness in one direction relative to other directions of the reinforcement. In the case of mechanical treatment, the mechanical treatment may comprise at least one of: grinding, finishing, abrading, inflating, shrinking, directionally-specific shrinking, inducing tension, stacking, coating, or expanding.

Providing an anisotropic reinforcement with selective reinforcement may also comprise reinforcing said anisotropic reinforcement with at least one of: additional fibers, natural fibers, synthetic fibers, mesh, collagen fibers, metals, cloth, or biocompatible metals. In the case of biocompatible metals, the biocompatible metals may be selected front stainless steel, titanium, metal alloys, or a combination thereof. In the case of metal alloys, the metal alloys may be selected from: In—Ti, Fe—Mn, Ni—Ti, Ag—Cd, Au—Cd, Au—Cu, Cu—Al—Ni, Cu—Au—Zn, Cu—Zn, Cu—Zn—Al, Cu—Zn—Sn, Cu—Zn—Xe, Fe₃Be, Fe₃Pt, Ni—Ti—V, Fe—Ni—Ti—Co, or Cu—Sn. The anisotropic reinforcement may also be a synthetic material, selected from tantalum gauze, stainless steel mesh, DACRON, ORLON, FORTISAN, nylon, knitted polypropylene (MARLEX), microporous expanded-polytetrafluoroethylene (GORE-TEX), Dacron-reinforced silicone rubber (SILASTIC), polyglactin 910 (VICRYL), polyester (MERSILENE), polyglycolic acid (DEXON), or a combination thereof.

According to an embodiment, communicating said anisotropic reinforcement with the heart may comprise at least one of adhesion, attachment, or suture. According to an embodiment, the infarctions may heal while resisting circumferential stretching, and may deform normally in the longitudinal and radial directions during myocardial contractions. According to an embodiment the infarctions may heal while resisting longitudinal stretching, and ma deform normally in the circumferential and radial directions during myocardial contractions.

Available synthetic patches can also be modified or newly engineered or fabricated to attain stiffness in one orientation in the reinforcement versus other orientations by preferentially adding fibers to the patch in one direction versus other directions to increase stiffness in the one direction versus the other directions. In this embodiment, a greater number of fibers, (e.g., a number greater than one), or a plurality of fibers, comprises one direction of the reinforcement relative to the number of fibers comprising other directions. The plurality of fibers, all oriented in one direction, affords the stiffness and greater rigidity to the reinforcement in the one direction of orientation, e.g., the longitudinal direction, versus other directions, e.g., the circumferential, latitudinal, or radial directions, of the patch. This provides the anisotropy that achieves improved healing and functioning of a repaired cardiac defect, incision, opening, or infarct. The stiffness in one direction of the reinforcement can be produced by using more of the same fibers or material as used in the original patch, or by using another, or different, synthetic fiber or material that is added to the reinforcement and oriented in the one direction of the patch. Natural fibers or materials, such as collagen fibers, can also be added to a reinforcement to increase the stiffness in the one direction of the reinforcement versus other directions.

The size of the anisotropic reinforcements according to an embodiment can be determined by the skilled practitioner. Reinforcement size is typically related to the ultimate type of use for the reinforcement and to the size of the opening, incision, defect, deformity, infarct, and the like, which is undergoing repair, augmentation, or restoration. Suitably sized anisotropic reinforcements can be utilized.

In one embodiment, a reinforcement may be reinforced in one direction versus other directions using other or different biocompatible materials, thereby making the reinforcement stiffer or more rigid in the one direction. Preferably, the material is approved for use in the body. Such reinforcing materials can include any material that is biocompatible and that is generally firmer, or more rigid and taut, than the reinforcement material itself. The reinforcing material can also comprise more of the original reinforcement material that is added to the patch, resulting in stiffness in one direction. Nonlimiting examples of reinforcing materials also include another type of synthetic material or small metal wire materials. Illustratively and without limitation, such metal materials include stainless steel, titanium and metal alloys. In addition, materials with shape memories work well for this purpose, as do combinations of materials that provide a shape memory. For example, the reinforcing material can be fabricated from superelastic materials comprising metal alloys.

Superelastic materials can comprise metal alloys of, but not limited thereto, the following: In—Ti, Fe—Mn, Ni—Ti, Ag—Cd, Au—Cd, Au—Cu, Cu—Al—Ni, Cu—Au—Zn, Cu—Zn, Cu—Zn—Xe, Fe₃Be, Fe₃Pt, Ni—Ti—V, Fe—Ni—Ti—Co, and Cu—Sn. One superelastic material that can be used comprises a nickel and titanium alloy, known commonly as nitinol (available from Memry Corp., Brookfield, Conn., or SMA Inc., San Jose, Calif.). The ratio of nickel and titanium in nitinol may be varied. Examples include a ratio of about 50% to about 52% nickel by weight, or a ratio of about 47% to about 49% nickel by weight. Nitinol has shape retention properties in its superelastic phase.

An embodiment, encompasses a method of producing an anisotropic reinforcement comprising weaving the angles of the fibers comprising a synthetic patch, such as a DACRON® patch, so that the angles of the fibers in one orientation of the reinforcement are smaller than the angles of the fibers in other orientations of the patch. This produces a stiffness or rigidity of those fibers in the one orientation of the reinforcement relative to the fibers in other orientations of the patch. In this embodiment, an anisotropic reinforcement is produced in which the fibers are stiffer or more rigid in one orientation of the weave of the patch, while the fibers in other directions of the weave are not particularly stiff or rigid. As a nonlimiting example, the fibers of the weave that are stiffer or more rigid in the reinforcement are oriented within about 10° to less than about 90°, or about 20° to about 70°, or about 25° to about 50°, or about 30° to about 45°, or about 30° of the local circumferential axis. The resulting anisotropic reinforcement allows a repaired cardiovascular defect, opening, incision, and the like, to heal while resisting circumferential stretching, yet deforms normally in the longitudinal and radial directions during myocardial contractions.

An embodiment encompasses a method of producing an anisotropic reinforcement comprising adding to a synthetic patch, e.g., a DACRON® patch, more fibers, or a biocompatible reinforcing material, oriented in a single direction in the patch. The reinforcing material is typically stiffer than the existing reinforcement material and can encompass, for example, additional or different fibers or fiber material, either natural or synthetic, or small metal wire materials, such as stainless steel, titanium and metal alloys, e,g., nitinol. In this embodiment, an anisotropic reinforcement is produced in which the stiffer and/or reinforcing material is oriented in one direction of the reinforcement resulting in stiffness in the one direction. Illustratively, the stiffer and/or reinforcing material is oriented in one direction relative to the circumference or radial directions of the patch. An aspect of a related embodiment embraces a method of preparing an anisotropic reinforcement involving adding externally to a synthetic patch biocompatible reinforcing material oriented in a single direction of the patch. The biocompatible reinforcing material is stiffer than the existing reinforcement material and creates a stiffness to the reinforcement in the single direction of the reinforcement relative to other directions of the patch.

An aspect of an embodiment encompasses a method of producing an anisotropic reinforcement comprising creating small slits, cuts, or openings in a synthetic patch, e.g., DACRON® patch. According to the method, the slits, cuts, or openings are made along one direction of the reinforcement so that after placement over an opening, incision, or infarct in the heart, for example, the reinforcement softens selectively in the direction perpendicular to the slits, cuts, or openings. Illustratively, if parallel slits are made in the longitudinal direction of a patch, such as a commercially-available DACRON® patch, an anisotropic reinforcement is created in which the reinforcement stretches more in the direction perpendicular to the slits and less in the longitudinal direction comprising the stiffness. In one embodiment, there can be at least one slit in the material, or there can be any number of slits to result in the desired mechanical properties, including and not limited to 100 slits or more.

An embodiment encompasses new and useful products. As described hereinabove, these products are reinforcements comprising fibers, weave, mesh, or otherwise interlaced or networked components, which are oriented in one predominant direction in the patch. Such anisotropic reinforcements are well suited for cardiovascular repair and are configured to resist high circumferential stresses while allowing freedom of longitudinal and radial deformation in adjacent regions of the myocardium, such as non-infarcted myocardium. In an aspect of an embodiment the reinforcements and materials are designed to parallel the anisotropic collagen fiber orientation, e.g., circumferentially around the heart, that is observed to occur in scar tissue following cardiovascular defect repair and post-infarction healing in order to minimize stress and pressure on the healing myocardium.

An embodiment embraces a variety of anisotropic reinforcements. One embodiment is directed to an anisotropic reinforcement comprising fibers oriented in one direction of the reinforcement to create stiffness in the one direction relative to other directions of the patch. In an embodiment, the fibers oriented in the one direction of the reinforcement comprise a plurality of fibers relative to the fibers in other directions of the patch. In an embodiment, the fibers in the one direction of the reinforcement are oriented in a line (a straight line) relative to non-linear, randomly placed, or coiled fibers in other directions of the patch. In an embodiment, the spacing of the pore sizes within the fibers in the one direction of the reinforcement is smaller than the spacing of the pore sizes of the fibers within other directions of the reinforcement so that the pores in the one direction are in closer proximity to each other than are the pores in other directions of the patch. In an embodiment, the fibers oriented in the one direction of the reinforcement are denser or thicker than the fibers in other directions of the patch. In an embodiment, the fibers oriented in the one direction of the reinforcement are reinforced in the one direction relative to the fibers in other directions of the patch. The reinforcement can include one or more different fibers and/or one or more biocompatible metals, which can be selected from stainless steel, titanium, metal alloys, or a combination thereof. Particular metal alloys can include, without limitation, In—Ti, Fe—Mn, Ni—Ti, Ag—Cd, Au—Cd, Au—Cu, Cu—Al—Ni, Cu—Au——Zn, Cu—Zn, Cu—Zn—Al, Cu—Zn—Sn, Cu—Zn—Xe, Fe₃Be, Fe₃Pt, Ni—Ti—V, Fe—Ni—Ti Co, or Cu—Sn. In an embodiment, the fibers can comprise collagen or synthetic mesh. Particular synthetic materials of which the reinforcement can be created include, without limitation, tantalum gauze, stainless steel mesh, DACRON®, ORLON®, FORTISAN®, nylon, knitted polypropylene (MARLEX®), microporous expanded-polytetrafluoroethylene (GORE-TEX®), dacron-reinforced silicone rubber (SILASTIC®), polyglactin 910 (VICRYL®), polyester (MERSILENE®), polyglycolic acid (DEXON®), or a combination thereof. Illustratively, DACRON® and GORE-TEX® (e.g., GORE-TEX® Acuseal Cardiovascular Patch) are especially suitable reinforcement materials.

An embodiment is directed to an anisotropic reinforcement comprising fibers aligned in a single direction for increased stiffness of the reinforcement in the direction of fiber alignment relative to directions of fiber nonalignment. In an embodiment, a plurality of aligned fibers comprises the single direction of the reinforcement to achieve increased stiffness relative to the number of fibers in other directions of the patch. In an embodiment, the fibers aligned in the single direction of the reinforcement are of larger size relative to the size of the fibers in other directions of the patch. In an embodiment, the fibers aligned in the single direction of the reinforcement are reinforced in the single direction of the reinforcement relative to the fibers in other directions of the patch. In an embodiment, the reinforcement comprises one or more of the same or different fibers, either natural or synthetic, or one or more biocompatible metals, or a combination thereof, as described above. In an embodiment, the fibers can be composed of collagen or of a synthetic material as described above.

An embodiment is directed to an anisotropic reinforcement comprising interwoven fibers, wherein a plurality of fibers is oriented in a single direction of the reinforcement to produce increased stiffness in the single direction relative to other directions perpendicular thereto. In an embodiment, the plurality of fibers is woven in the longitudinal direction of the reinforcement relative to the latitudinal (circumferential) and radial directions of the patch. In an embodiment, added fibers, either the same as or different from the original reinforcement material, are woven into the reinforcement in the one direction of the reinforcement to produce stiffness in the one direction relative to other directions without added fibers.

An embodiment is directed to an anisotropic reinforcement comprising longitudinal fibers oriented in a single direction to produce stiffness in the longitudinal direction relative to fibers in the latitudinal (circumferential) and radial directions of the patch. In an embodiment, the longitudinal fibers comprise a plurality of fibers creating stiffness in the longitudinal direction relative to fibers in the latitudinal and radial directions of the patch. In an embodiment, the longitudinal fibers comprise larger fibers creating stiffness in the longitudinal direction relative to smaller fibers in the latitudinal and radial directions of the patch. In an embodiment, the longitudinal fibers comprise denser or thicker fibers creating stiffness in the longitudinal direction relative to less dense or thick fibers in the latitudinal (circumferential) and radial directions of the patch. In an embodiment, the longitudinal fibers comprise smaller pore sizes creating stiffness in the longitudinal direction relative to larger pore sizes of the fibers in the latitudinal (circumferential) and radial directions of the patch. In an embodiment, the longitudinal fibers are reinforced to create stiffness in the longitudinal direction relative to unreinforced fibers in the latitudinal (circumferential) and radial directions of the patch. In an embodiment, the reinforcement comprises one or more of the same or different fibers, either natural or synthetic, one or more biocompatible metals, or a combination thereof, as described above.

An embodiment is directed to an anisotropic reinforcement comprising fibers, which are aligned in a single direction resulting in an increased stiffness of the reinforcement in the direction of fiber alignment relative to the directions of fiber non-alignment. In an embodiment, the fibers are aligned longitudinally in the single direction to result in increased stiffness in the longitudinal direction. In an embodiment, the fibers are aligned in the single direction along a vertical axis. In an embodiment, the fibers are composed of collagen or synthetic mesh. In an embodiment, the reinforcement is composed of a synthetic material as described above. In an embodiment, the reinforcement further contains the same or different added fibers, or biocompatible metal wire, for example, stainless steel, titanium, metal alloys, or a combination thereof, to enhance stiffness in the single direction. Of particular interest is a nickel-titanium alloy called nitinol as described above.

The anisotropic reinforcements of an embodiment are intended for surgical use for both non-human mammals, such as in veterinary medicine, as well as for human patients. For ease of use, in an embodiment the anisotropic reinforcements and synthetic materials ideally contain a marking thereon to establish the orientation in which they should be placed during surgery. For example, when used in heart surgery, a reinforcement can be placed such that the stiffer direction of the reinforcement is aligned, for example, with the circumference of the heart, or in the longitudinal direction of the heart. In addition, the product, package or packing label and/or instructions for the anisotropic reinforcements can include information to the surgeon or skilled practitioner regarding proper placement of the reinforcement during surgery. For example, the instructions can include information for the surgeon to align the stiffer direction or orientation of the anisotropic reinforcement around the circumference, or in the longitudinal direction, of an incision, opening, defect, and the like, that is undergoing repair.

The anisotropic reinforcements and synthetic materials according to an embodiment can be used in the repair, restoration, or amelioration of a lumen comprising another type of anatomical vessel or passageway of the body, e.g., a bile duct, the lumen of the gut, in addition to blood vessels, arteries, aortic vessels. In this embodiment, the reinforcements can be used in connection with the insertion of a stent into the vessel, duct, or lumen, for example.

An embodiment encompasses a method of repairing, reinforcing, or ameliorating an opening, defect, wound, incision, and the like, in a mechanically anisotropic tissue, e.g., skin, tendon, gut, intestine, or muscle wall. The method comprises implanting over the opening, defect, wound, incision, and the like, an anisotropic reinforcement as described herein. An aspect of an embodiment of is directed to a method of repairing, reinforcing, or ameliorating a cardiovascular incision or opening, comprising implanting over the incision or opening an anisotropic reinforcement as described herein. An aspect of an embodiment is directed to a method of repairing, reinforcing, or ameliorating a myocardial incision or opening, comprising implanting over the myocardial incision or opening an anisotropic reinforcement as described herein. An embodiment is directed to a method of repairing, reinforcing, or ameliorating a blood vessel or aortic vessel incision or opening, comprising implanting over the blood vessel or aortic vessel incision or opening an anisotropic reinforcement as described herein. The anisotropic reinforcements of an embodiment are typically used during open-heart surgery or other cardiovascular surgical procedures. As used herein, implanting generally refers to inserting, placing, or positioning a reinforcement of an embodiment to cover an incision or opening and the like, as would be understood by the skilled practitioner in the art. Thereafter, the reinforcement is secured at the site, such as by suturing, to remain in place during healing and recovery following surgery.

In general, during implantation and use, the three dimensional orientation of an anisotropic reinforcement as described herein is such that the stiffer direction of the reinforcement is aligned with the circumference of the heart, or around the circumference of the lumen or vessel, or with the axis of greatest stiffness of the neighboring normal tissue.

An embodiment encompasses a method of strengthening a weakness in a body or muscle wall comprising applying an anisotropic reinforcement as made or described herein in the area of the body or muscle wall weakness. In an embodiment, the opening, defect, wound, or incision in the body or muscle wall comprises a hernia. An embodiment encompasses a method of strengthening a weakness in myocardial tissue, e.g., the heart, comprising applying an anisotropic reinforcement as made or described herein in the area of the myocardial tissue weakness. An embodiment encompasses a method of strengthening a weakness in a vessel or passageway in the body, for example, a blood vessel, an artery, an aortic vessel, a bile duct, a genitourinary tract vessel or duct, or a gastrointestinal vessel or duct, etc., which involves applying an anisotropic reinforcement as made or described herein in the area of vessel weakness.

### EXAMPLES AND EXPERIMENTAL RESULTS

Practice of an aspect of an embodiment (or embodiments) may be more fully understood from the following examples and experimental results, which are presented herein for illustration only and should not be construed as limiting the invention in any way.

### Example and Experimental Results Set No. 1

Evidence that Selective Reinforcement of Scar Will Improve Heart Function:

A series of computational modeling studies were conducted to assess the effect of varying scar mechanical properties on left ventricular function. Ventricular function was assessed using the end-systolic pressure-volume relationship (ESPVR). This indicates the volume remaining in the heart at the end of ejection under a range of different loading conditions. Loss of contracting muscle during a heart attack may shift this curve to the right—the heart is now capable of ejecting less blood against any pressure, so a larger volume remains in the heart at the end of ejection. We studied whether making the scar tissue stiffer in one direction would shift the ESPVR leftward, back towards normal.

As shown in FIG. 7, when we simulated a particular infarct—a large infarct on the anterior wall of the heart, we found that circumferential reinforcement resulted in modest improvement, but longitudinal reinforcement produce a much greater improvement (FIG. 7C). Local patterns of stretching revealed the reason that longitudinal reinforcement was more effective: without reinforcement, this particular infarct stretched dramatically in the longitudinal direction while the rest of the heart was contracting (FIG. 7A), but stretched little in the circumferential direction (not shown). Therefore, circumferential reinforcement did not change the infarct deformation much, while longitudinal reinforcement greatly reduced longitudinal stretching.

As shown in FIG. 7, modeling results supporting longitudinal reinforcement of large antero-apical infarcts in the dog. In FIG. 7A a map of longitudinal strain in a simulated infarct shows dramatic stretching in the longitudinal direction (>20%, white region in center of plot). By contrast, simulations predicted little stretching in the circumferential direction (<4%). In FIG. 7B it is graphically shown that selectively reinforcing the infarct in the longitudinal direction greatly reduced stretching. In FIG. 4 it is graphically shown that the longitudinal reinforcement improved systolic function more than circumferential reinforcement, as reflected in a leftward shift of the end-systolic pressure-volume relationship (‘infarct’=acute infarct, ‘circ’=circumferential reinforcement, ‘long’=longitudinal reinforcement.)

Additional modeling studies have revealed that simulated infarcts in different locations experienced very different loads, suggesting that clinical application of infarct reinforcement will need to be tailored to individual patients or at least to each common infarct location. This very interesting finding may prove an important part of the intellectual property surrounding infarct reinforcement, as mentioned above.

### Example and Experimental Results Set No. 2

Evidence that Selective Reinforcement of Scar has the Predicted Effect:

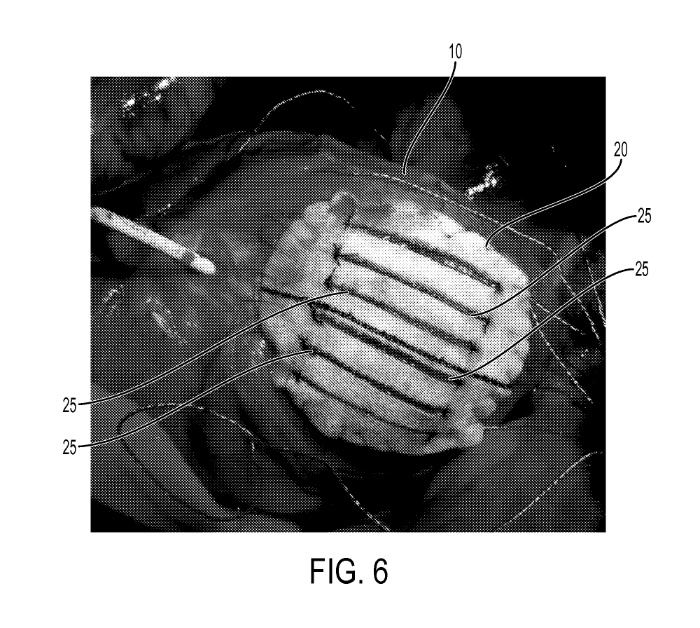

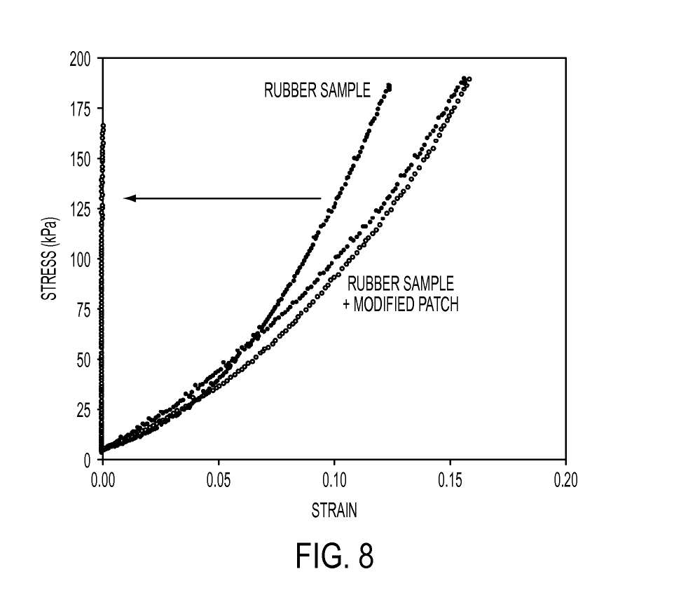

Following completion of the modeling studies described above, we established a method for modifying commercially available Dacron patches (Hemashield, Boston Scientific) so that they are very stiff in one direction but offer little resistance to deformation. Accordingly, this result is graphically shown in FIG. 8. Regarding this experiment, we began a series of acute large-animal studies where we instrument the heart to measure pressure, volume, and local deformation; ligate a coronary artery to create an experimental infarction; and then sew a modified patch 20 to the epicardial surface of the heart 9 (FIG. 6). Before and after applying the patch, we measure global and regional function to assess the impact of the patch. As part of the process, we cut parallel slits in the patches to weaken them in the direction perpendicular to the slits; they remain very stiff in the direction parallel to the slits. We then created large antero-apical myocardial infarcts in open-chest dogs, waited 60 minutes for the infarct to take full effect, and sewed the modified patch to the epicardial surface. We blocked reflex changes in heart rate or contractility and compared pump function at matched filling pressures.

As shown in FIG. 8, infarct reinforcement with a modified Dacron patch. We modified a Boston Scientific Hemashield patch by cutting slits in one direction. As shown in FIG. 8 it is evidenced that sewing this patch to an isotropic rubber sample reinforced it in just one direction (as shown as vertical line of points along the stress axis), without altering stiffness in the other direction. FIG. 6 illustrates a photographic depiction of a dog's heart 10 and the reinforcement 20. As shown in FIG. 6 we then sewed modified patches 20 having slits or elongated apertures 25, to the epicardial surface in two dogs following coronary occlusion (white tube to L of patch is occluder).

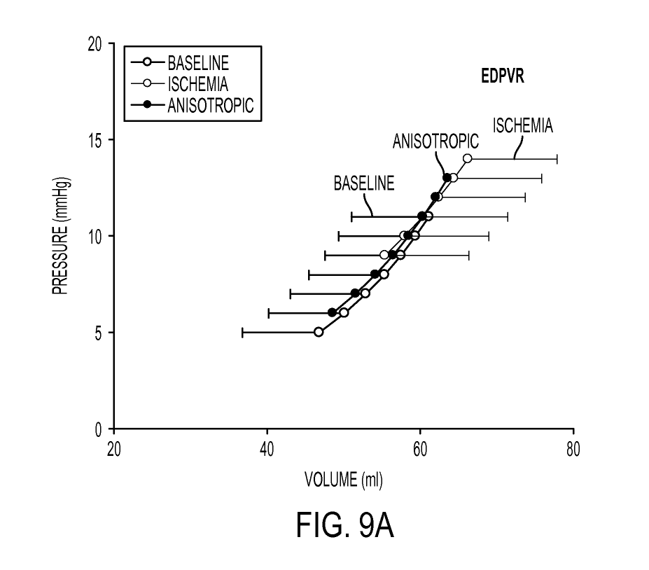

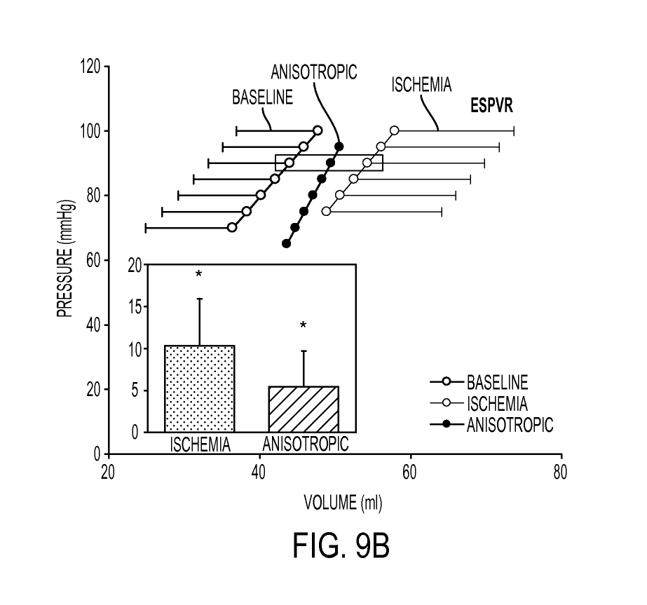

As graphically shown in FIG. 9, on average in five dogs, referring to the pressure-volume curve (FIG. 9A), diastolic function was not changed by ischemia or by reinforcement, while reinforcement did return systolic function halfway back to normal. (FIG. 9B).

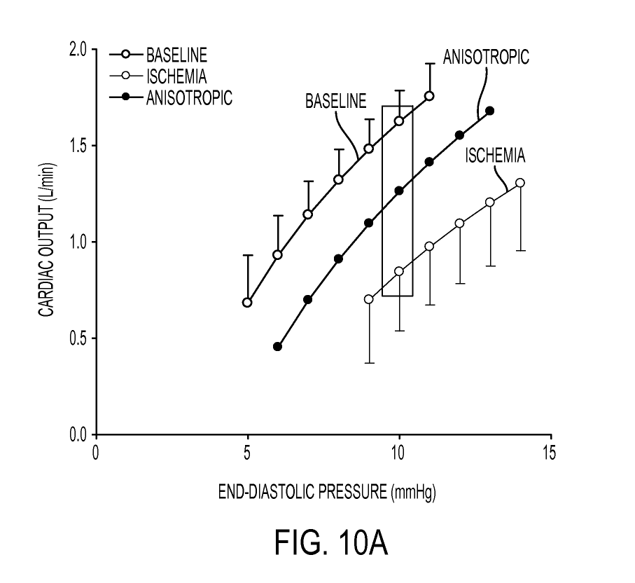

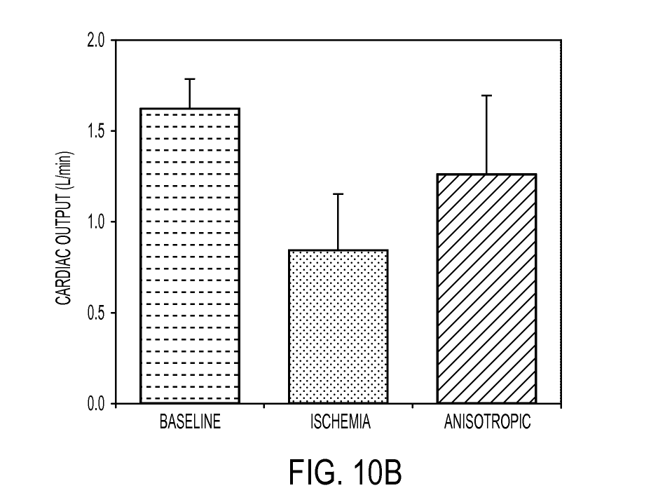

As graphically shown in FIG. 10, consistent with the ability of reinforcement to improve systolic function without altering diastolic function, cardiac output curves confirm that ischemia dramatically depresses pump function, reducing cardiac output by 50% at an end-diastolic pressure of 10 mmHg. Reinforcement rescues half of the deficit in the cardiac output curve and in cardiac output at a filling pressure of 110 mmHg.

### Example and Experimental Results Set No. 3

In a pig model, we found that the scar that forms eventually reinforces itself in the right direction (it gets stiffer in the direction that has the most stretch, reducing that stretch). However, the scar formation process takes weeks, and much of the remodeling of the scar and border region that ultimately starts a patient on the road to heart failure happens in the first few days. Therefore, we submit it is important to intervene early, before too much remodeling has occurred, to modify mechanics of the damaged region and try to prevent the process of remodeling and progression to heart failure.

The devices, systems, compositions, techniques, designs and methods of various embodiments of the invention disclosed herein may utilize aspects disclosed in the following references, applications, publications and patents and which are hereby incorporated by reference herein in their entirety:

1. U.S. Patent Application Publication No. 2008/0009830 A1, “Biogradable Elastomeric Patch for Treating Cardiac or Cardiovascular Conditions”, Fujimoto, et al., Jan. 10, 2008.

2. U.S. Pat. No. 6,544,167 B2, “Ventricular Restoration Patch”, Buckberg, et al., Apr. 8, 2003.

3. U.S. Application Publication No. 2005/0125012 A1, “Hemostatic Patch for Treating Congestive Heart Failure”, Houser, et al., Jun. 9, 2005.

4. U.S. Pat. No. 6,685,620, “Ventricular Infarct Assist Device and Methods for Using It”, Gifford, I I I, et al., Feb. 3, 2004.

5. U.S. Pat. No. 4,552,707, “Synthetic Vascular Grafts, and Methods of Manufacturing Such Grafts”, How, T., Nov. 12, 1985.

6. U.S. Pat. No. 7,364,587, “High Stretch, Low Dilation Knit Prosthetic Device and Method for Making the Same”, Dong, et al., Apr. 29, 2008.

7. U.S. Patent Application Publication No. US2008/0091057, A1, Walker, J., “Method and Apparatus for Passive Left Atrial Support”, Apr. 17, 2008.

8. U.S. Pat. No. 7,361,137 B2, Taylor, et al., “Surgical Procedures and Devices for Increasing Cardiac Output of the Heart”, Apr. 22, 2008.

9. U.S. Patent Application No. US 2008/0319308 A1, Tang, D., “Patient-Specific Image-Based Computational Modeling and Techniques for Human Heart Surgery Optimization”, Dec. 25, 2008.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

1. A reinforcement for communication with the heart, wherein said reinforcement is configured to create stiffness in one direction relative to other directions of said reinforcement, for reinforcing a region of the heart for improving heart function.

2. The reinforcement of claim 1, wherein said configuration is achieved by an attachment technique of said reinforcement to the heart.

3. The reinforcement of claim 1, wherein said configuration is provided whereby said reinforcement has said configuration prior to said reinforcement attached to said heart.

4. The reinforcement of claim 1, wherein said configuration is provided by both of:
an attachment technique of said reinforcement to the heart; and
as said configuration is provided prior to the attachment technique to the heart.

5. The reinforcement of claim 1, wherein said heart function comprises pump function.

6. The reinforcement of claim 1, wherein said heart function comprises at least one of the following: systolic function or contraction of the heart.

7. The reinforcement of claim 1, wherein said improving heart function comprises resisting longitudinal stretching of the region of the heart.

8. The reinforcement of claim 7, wherein said resisting longitudinal stretching of the region of the heart occurs during myocardial contractions.

9. The reinforcement of claim 7, wherein said improving heart function further comprises allowing normal circumferential and radial deformation of the region of the heart.

10. The reinforcement of claim 1, wherein said improving heart function comprises resisting circumferential stretching of the region of the heart.

11. The reinforcement of claim 10, wherein said resisting circumferential stretching of the region of the heart occurs during myocardial contractions.

12. The reinforcement of claim 10, wherein said improving heart function further comprises allowing normal longitudinal and radial deformation of the region of the heart.

13. The reinforcement of claim 1, wherein said heart function comprises at least one of the following: cardiac output, ejection fraction, volumes, stroke volume, pressures, end-diastolic volume (EDV), end-systolic volume (ESV), energetics, energetic efficiency, and need for inotropic support.

14. The reinforcement of claim 1, wherein said region of the heart comprises at least one of the following: at least a portion of a wall, at least a portion of an ischemic, at least a portion of an infarct, at least a portion of an epicardial surface, and at least a portion of an inner surface.

15. The reinforcement of claim 1, wherein said communication comprises at least one of: adhesion, attachment, or suture.

16. The reinforcement of claim 1, wherein said reinforcement comprises at least one of: graft, patch, member, local-reinforcement, substrate, material, wire, local-reinforcing member, members applied to the heart, members into the heart, support, brace, buttress, coating, augmentation, and fortification.

17. The reinforcement of claim 1, wherein said reinforcement comprises a patch with at least substantially parallel slit apertures or elongated apertures in said patch to decrease stiffness of the patch in the direction at least substantially perpendicular to said slit apertures or elongated apertures.

18. The reinforcement of claim 1, wherein said reinforcement provides flexibility in the one direction at least substantially perpendicular to the stiffness.

19. The reinforcement of claim 1, wherein said reinforcement comprises fibers oriented in the one direction of said reinforcement to create stiffness in the one direction relative to fibers other directions of said reinforcement.

20. The reinforcement of claim 19 wherein said fibers oriented in the one direction of said reinforcement comprise a plurality of fibers relative to said fibers in the other directions of said reinforcement.

21. The reinforcement of claim 19, wherein said fibers in the one direction of said reinforcement are oriented in at least a substantially straight line relative to randomly or stochasticly placed fibers in other directions of said reinforcement.

22. The reinforcement of claim 19, wherein said fibers in the one direction of said reinforcement are tight relative to fibers in other directions of said reinforcement.

23. The reinforcement of claim 19, wherein the fibers in the one direction of said reinforcement arc less slack relative to fibers in other directions of said reinforcement.

24. The reinforcement of claim 19, wherein pores or apertures within the fibers in the one direction of said reinforcement are in closer proximity to each other than pores or apertures within said fibers in other directions of said reinforcement.

25. The reinforcement of claim 19, wherein said fibers oriented in the one direction of said reinforcement are denser relative to said fibers in other directions of said reinforcement.

26. The reinforcement of claim 19, wherein said fibers oriented in the one direction of said reinforcement are locally-reinforced in the one direction relative to said fibers in other directions of said reinforcement.

27. The reinforcement of claim 26, wherein said fiber local-reinforcement comprises at least one of: additional fibers, natural fibers, synthetic fibers, mesh, collagen fibers, metals, wires, cloth, biocompatible metals, or a combination thereof.

28. The reinforcement of claim 27, wherein said biocompatible metals may comprise at least one of: stainless steel, titanium, metal alloys, or a combination thereof.

29. The reinforcement of claim 28, wherein the metal alloys may comprise at least one of: In—Ti, Fe—Mn, Ni—Ti, Ag—Cd, Au—Cd, Au—Cu, Cu—Al—Ni, Cu—Au—Zn, Cu—Zn, Cu—Zn—Al, Cu—Zn—Sn, Cu—Zn—Xe, Fe₃Be—Fe₃Pt, Ni—Ti—V, Fe—Ni—Ti—Co, Cu—Sn or a combination thereof.

30. The reinforcement of claim 1, wherein said reinforcement comprises a synthetic material.

31. The reinforcement of claim 30, wherein said synthetic material may comprise at least one of tantalum gauze, stainless steel mesh, DACRON, ORLON, FORTISAN, nylon, knitted polypropylene (MARLEX), microporous expanded-polytetrafluoroethylene (GORE-TEX), Dacron-reinforced silicone rubber (SILASTIC), polyglactin 910 (VICRYL), polyester (MERSILENE), polyglycolic acid (DEXON), or a combination thereof.

32. The reinforcement of claim 1, wherein said stiffness of said reinforcement is configured to be aligned in a substantially longitudinal direction of the heart.

33. The reinforcement of claim 1, wherein said stiffness of said reinforcement is configured to be aligned in a substantially circumferential direction of the heart.

34. The reinforcement of claim 1, wherein said stiffness of said reinforcement is configured to be at least substantially aligned with the underlying muscle fiber direction of the heart and/or collagen fiber direction of said infarct region.

35. The reinforcement of claim 1, wherein said stiffness of said reinforcement is configured to be aligned at least substantially transverse with the underlying muscle fiber direction of the heart and/or collagen fiber direction of said infarct region.

36. The reinforcement of claim 1, wherein said stiffness of said reinforcement is configured to be aligned with the direction of greatest stretching of the region of the heart.

37. The reinforcement of claim 1, wherein said reinforcement comprises fibers aligned in at least a substantially single direction for increased stiffness of said reinforcement in the direction of fiber alignment relative to directions of fiber nonalignment.

38. The reinforcement of claim 37, wherein the fibers are aligned longitudinally in said at least substantially single direction in said reinforcement to result in increased stiffness in the longitudinal direction.

39. The reinforcement of claim 37, wherein said fibers are aligned in said at least substantially single direction is along said reinforcement's longitudinal axis.

40. The reinforcement of claim 37, wherein said fibers aligned in said at least substantially single direction are a larger size relative to the size of said fibers in other directions of said reinforcement.

41. The reinforcement of claim 37, wherein said fibers aligned in said at least substantially single direction are locally-reinforced in said at least substantially single direction relative to said fibers in other directions of said reinforcement.

42. The reinforcement of claim 1, wherein said reinforcement comprises interwoven fibers, wherein a plurality of said interwoven fibers are oriented at least substantially in a single direction within said reinforcement to produce increased stiffness in said at least substantially single direction relative to other directions.

43. The reinforcement of claim 42, wherein said other directions include at least substantially perpendicular, transverse or diagonal thereto.

44. The reinforcement of claim 42, wherein the plurality of fibers are oriented in the longitudinal direction of said reinforcement relative to fibers in the substantially circumferential, radial, perpendicular, or diagonal directions of said reinforcement.

45. The reinforcement of claim 42, wherein the plurality of fibers are the same number and/or material as the fibers comprising said reinforcement.

46. The reinforcement of claim 42, wherein the plurality of fibers are different in number and/or material from the fibers comprising said reinforcement.

47. The reinforcement of claim 1, wherein said reinforcement comprises strips of a stiffer material relative to at least some non-strip areas, wherein said strips are configured for attachment to the region of the heart such that the longitudinal axis of said strips are oriented in a direction desirable for reinforcing the heart.

48. The reinforcement of claim 47, wherein said strips are integrally connected and/or separate from one another.

49. The reinforcement of claim 47, wherein said stiff material comprises cardiovascular fabrics.

50. The reinforcement of claim 47, wherein said longitudinal axis of said strips are configured to be aligned in a substantially longitudinal direction of the heart.

51. The reinforcement of claim 47, wherein said longitudinal axis of said strips are configured to be aligned in a substantially circumferential direction of the heart.

52. The reinforcement of claim 47, wherein said longitudinal axis of said strips are configured to be at least substantially aligned with the underlying muscle fiber direction of the heart and/or collagen fiber direction of said infarct region.

53. The reinforcement of claim 47, wherein said longitudinal axis of said strips are configured to be aligned at least substantially transverse with the underlying muscle fiber direction of the heart and/or collagen fiber direction of said infarct region.

54. The reinforcement of claim 1, wherein at least a portion of said reinforcement is stiff relative to other portions of said reinforcement to create at least one stiff regions substantially in one direction of said reinforcement.

55. The reinforcement of claim 54, wherein said reinforcement is anisotropic.

56. The reinforcement of claim 54, wherein said at least one relative stiff portion is tight relative to other regions of reinforcement.

57. The reinforcement of claim 54, wherein said at least one relative stiff portion is less slack relative to said other portions of said reinforcement.

58. The reinforcement of claim 54, wherein said at least one relative stiff portion is denser relative to said other portions of said reinforcement.

59. The anisotropic reinforcement of claim 54, wherein said at least one relative stiff portion is further locally-reinforced relative to said other portions of said reinforcement.

60. The anisotropic reinforcement of claim 59, wherein said local reinforcement comprises at least one of: fibers, additional fibers, natural fibers, synthetic fibers, mesh, collagen fibers, metals, cloth, wires, fabric, braid, or biocompatible metals.

61. The reinforcement of claim 60, wherein the biocompatible metals may comprise at least one of stainless steel, titanium, metal alloys, or a combination thereof.

62. The reinforcement of claim 61, wherein the metal alloys may comprise at least one of In—Ti, Fe—Mn, Ni—Ti, Ag—Cd, Au—Cd, Au—Cu, Cu—Al—Ni, Cu—Au—Zn, Cu—Zn, Cu—Zn—Al, Cu—Zn—Sn, Cu—Zn—Xe, Fe₃Be, Fe₃Pt, Ni—Ti—V, Fe—Ni—Ti—Co, Cu—Sn or a combination thereof.

63. The reinforcement of claim 54, wherein said at least one relatively stiff portion is aligned longitudinally in at least a substantially single direction in said reinforcement to result in increased stiffness in the longitudinal direction of said reinforcement.

64. The reinforcement of claim 54, wherein at least one relatively stiff portion is aligned in at least a substantially single direction along said reinforcement's longitudinal axis, relative to the said other portions of said reinforcement.

65. The reinforcement of claim 64, wherein said other portions include at least substantially perpendicular, transverse or diagonal regions of said reinforcement.

66. The reinforcement of claim 54, wherein at least one relatively stiff portion is oriented in the longitudinal direction of said reinforcement relative to regions in the substantially circumferential, radial, perpendicular, or diagonal directions of said reinforcement.

67. The anisotropic reinforcement of claim 54, wherein said reinforcement provides flexibility in a direction at least substantially perpendicular to said at least one relatively stiff portions.

68. The reinforcement of claim 1, wherein said reinforcement is chemically treated to create anisotropy.

69. The reinforcement of claim 1, wherein said reinforcement is mechanically treated to create anisotropy.

70. The reinforcement of claim 69, wherein mechanical treatment comprises at least one of: grinding, finishing, abrading, inflating, shrinking, directionally-specific shrinking, inducing tension, slacking, coating, and expanding.

71. The reinforcement of claim 1, wherein said reinforcement comprises a material comprising at least one of: shape memory material or structure, pre-stressed material or structure, recoil material or structure, active recoil material or structure, pre-shaped material or structure, or a combination thereof.

72. The reinforcement of claim 71, wherein said shape memory material is nitinol

73. The reinforcement of claim 1, wherein fibers oriented in one direction of said reinforcement are distributed over a smaller range of angles to produce stiffness in a direction, relative to other directions have fibers distributed over a larger range of angles.

74. The reinforcement of claim 1, wherein said reinforcement comprises smaller alignment angles in one direction of said reinforcement to produce stiffness in the one direction relative to fibers having larger angles of alignment.

75. The reinforcement of claim 74, wherein the stiffness in the one direction of said reinforcement comprises fibers oriented having the alignment angles within about 10 degrees to less than about 90 degrees relative to the local circumferential axis of said reinforcement.

76. The reinforcement of claim 74, wherein the stiffness in the one direction of said reinforcement comprises fibers oriented having the alignment angles within about 20 degrees to about 70 degrees relative to the local circumferential axis of said reinforcement.

77. The reinforcement of claim 74, wherein the stiffness in the one direction of said reinforcement comprises fibers oriented having the alignment angles within about 25 degrees to about 50 degrees relative to the local circumferential axis of said reinforcement.

78. The reinforcement of claim 74, wherein the stiffness in the one direction of said reinforcement comprises fibers oriented having the alignment angles within about 30 degrees to about 45 degrees relative to the local circumferential axis of said reinforcement.

79. The reinforcement of claim 1, wherein said reinforcement is configured to provide at least one of: drug treatment, cellular therapy, pacing capabilities, stern cell therapy, or mechanical integrity.

80. A reinforcement for communication with a heart possessing an infarction, whereby said reinforcement is configured to create stiffness in one direction relative to other directions of said reinforcement, to preferentially reinforce one direction of the infarct region of the heart wall.

81. The reinforcement of claim 80, wherein said preferential reinforcement provides said stiffness in at least one direction of said reinforcement that is at least substantially aligned with the underlying muscle fiber direction of the heart and/or collagen fiber direction of said infarct region

82. The reinforcement of claim 80, wherein said preferential reinforcement provides said stiffness in at least one direction of said reinforcement that is at least substantially transverse with the underlying muscle fiber direction of the heart and/or collagen fiber direction of said infarct region.

83. The reinforcement of claim 80, wherein said preferential reinforcement provides said stiffness in at least one direction of said reinforcement that is at least substantially aligned with the longitudinal direction of the heart.

84. The reinforcement of claim 80, wherein said preferential reinforcement provides said stiffness in at least one direction of said reinforcement that is at least substantially aligned with the circumferential direction of the heart.

85. A method for improving heart function, said method comprising:
communicating
a reinforcement with the heart, wherein said reinforcement is configured to create stiffness in one direction relative to other directions of said reinforcement, for reinforcement of the wall of the heart for said improved pump function.

86. A method for improving heart function, said method comprising:
determining the direction to reinforce an infarction;
providing an anisotropic reinforcement with selective reinforcement for said determined direction; and
communicating said anisotropic reinforcement with the heart for reinforcing said infarction.

87. The method of claim 86, wherein said determining comprises a clinical assessment or medical practitioner assessment of the infarction.

88. The method of claim 86, wherein said determining comprises imaging the infarction.

89. The method of claim 88, wherein the imaging comprises assessment of infarct stretching.

90. The method of claim 88, wherein imaging comprises the use of at least one of: MRI, X-Ray, CAT Scan, or Ultrasound technology.

91. The method of claim 86, wherein said providing comprises weaving tight fibers in one direction relative to other directions of said reinforcement to produce stiffness in the one direction relative to other directions of said reinforcement.

92. The method of claim 91, wherein said providing further comprises weaving loose fibers in the other directions of the anisotropic reinforcement relative to the one direction.

93. The method of claim 86, wherein said providing comprises weaving dense fibers in one direction of the anisotropic reinforcement relative to other directions of the anisotropic reinforcement to produce stiffness in the one direction relative to other directions of the anisotropic reinforcement.

94. The method of claim 93, wherein said providing an further comprises weaving loose fibers in the other directions of the anisotropic reinforcement relative to the one direction.

95. The method of claim 86, wherein said providing comprises weaving straight, tight, or stretched fibers in a single direction of the anisotropic reinforcement relative to other directions of the anisotropic reinforcement to produce stiffness in the single direction, relative to other directions of the anisotropic reinforcement.

96. The method of claim 95, wherein said providing an anisotropic reinforcement with selective reinforcement further comprises weaving randomly or stochastically oriented fibers in the other directions of the anisotropic reinforcement relative to the one direction.

97. The method of claim 95, wherein said providing further comprises weaving slack or unstretched fibers in the other directions of the anisotropic reinforcement relative to the one direction.

98. The method of claim 97, wherein the slack or unstretched fibers comprise at least one of: coiled, curved, or zig-zag fibers.

99. The method of claim 86, wherein said providing comprises weaving small pores or apertures within fibers comprising one direction of the anisotropic reinforcement relative to other directions of the anisotropic reinforcement to create stiffness in the one direction relative to the other directions of the anisotropic reinforcement.

100. The method of claim 99, wherein said providing further comprises weaving larger pores or apertures in the other directions of the anisotropic reinforcement relative to the one direction of the anisotropic reinforcement.

101. The method of claim 86, wherein said providing comprises cutting slits or elongated apertures in said anisotropic reinforcement along the one direction of said anisotropic reinforcement so that said anisotropic reinforcement stiffens selectively in the direction parallel to said slits or apertures.

102. The method of claim 86, wherein said providing comprises chemically, treating said reinforcement to render it anisotropic, such to create stiffness in one direction relative to other directions of the anisotropic reinforcement.

103. The method of claim 86, wherein said providing comprises mechanically treating said reinforcement to render it anisotropic, such to create stiffness in one direction relative to other directions of said reinforcement.

104. The method of claim 103, wherein mechanical treatment comprises at least one of:
grinding, finishing, abrading, inflating, shrinking, directionally-specific shrinking, inducing tension, slacking, coating, or expanding.

105. The method of claim 86, wherein said providing comprises locally-reinforcing said anisotropic reinforcement.

106. The method of claim 105, wherein said local-reinforcement comprises at least one of: additional fibers, natural fibers, synthetic fibers, mesh, collagen fibers, metals, wires, cloth, or biocompatible metals.

107. The method of claim 106, wherein the biocompatible metals may comprise at least one of stainless steel, titanium, metal alloys, or a combination thereof.

108. The method of claim 107, wherein the metal alloys may comprise at least one of In—Ti, Fe—Mn, Ni—Ti, Ag—Cd, Au—Cd, Au—Cu, Cu—Al—Ni, Cu—Au—Zn, Cu—Zn, Cu—Zn—Al, Cu—Zn—Sn, Cu—Zn—Xe, Fe₃Be, Fe₃Pt, Ni—Ti—V, Fe—Ni—Ti—Co, Cu—Sn or a combination thereof.

109. The method of claim 86, wherein said anisotropic reinforcement is a synthetic material.

110. The method of claim 109, wherein said synthetic material may comprise at least one of tantalum gauze, stainless steel mesh, DACRON, ORLON, FORTISAN, nylon, knitted polypropylene (MARLEX), microporous expanded-polytetrafluoroethylene (GORE-TEX), Dacron-reinforced silicone rubber (SILASTIC), polyglactin 910 (VICRYL), polyester (MERSILENE), polyglycolic acid (DEXON), or a combination thereof.

111. The method of claim 86, wherein said communicating comprises at least one of adhering, attaching and suturing said anisotropic reinforcement with said heart.

112. The method of claim 86, wherein said infarctions heal while resisting circumferential
stretching, and deform normally in the longitudinal and radial directions during myocardial contractions.

113. The method of claim 86, wherein said infarctions heal while resisting longitudinal stretching, and deform normally in the circumferential and radial directions during myocardial contractions.

114. The method of claim 86, wherein said direction to reinforce the infarction is determined to be the longitudinal direction of the heart.

115. The method of claim 86, wherein said direction to reinforce the infarction is determined to be the circumferential direction of the heart.

116. The method of claim 86, wherein said direction to reinforce the infarction is determined to be a direction at least substantially aligned with the underlying muscle fiber direction of the heart and/or collagen fiber direction of said infarct region.

117. The method of claim 86, wherein said direction to reinforce the infarction is determined to be a direction at least substantially transverse with the underlying muscle fiber direction of the heart and/or collagen fiber direction of said infarct region.

118. The method of claim 86, further comprising providing information to a surgeon or skilled practitioner regarding proper placement of said reinforcement.

119. The method of claim 118, wherein said information is provided at one or more of the following locations: on a surface of said reinforcement; on packaging associated with said reinforcement; on a packing label associated with said reinforcement; and/or in a set of instructions associated with said reinforcement.

120. The method of claim 118, wherein said reinforcement provides stiffness in one direction relative to other directions of said reinforcement, and wherein said information instructs the surgeon or skilled practitioner to align said one direction of said reinforcement around the circumference of an incision, opening, or defect undergoing repair.

121. The method of claim 118, wherein said reinforcement provides stiffness in one direction relative to other directions of said reinforcement, and wherein said information instructs the surgeon or skilled practitioner to align said one direction of said reinforcement around the circumference of the heart.

122. The method of claim 118, wherein said reinforcement provides stiffness in one direction relative to other directions of said reinforcement, and wherein said information instructs the surgeon or skilled practitioner to align said one direction of said reinforcement in the longitudinal direction of an incision, opening, or defect undergoing repair.

123. The method of claim 118, wherein said reinforcement provides stiffness in one direction relative to other directions of said reinforcement, and wherein said information instructs the surgeon or skilled practitioner to align said one direction of said reinforcement in the longitudinal direction of the heart.

124. A method for improving heart function, said method comprising:
determining the direction to reinforce an infarction; and
configuring a reinforcement, said configuration in accordance with said determined direction,and for selectively reinforcing said infarction.

125. The method of claim 124, wherein said selective reinforcement is anisotropic.

126. The method of claim 125, wherein said determining comprises a clinical assessment or medical practitioner assessment of the infarction.

127. The method of claim 125, wherein said determining comprises imaging the infarction.

128. The method of claim 124, wherein said configuration comprises:
providing said reinforcement, wherein said reinforcement has anisotropic properties; and
communicating said reinforcement so as to further provide additional anisotropic properties.

129. The method of claim 128, wherein said communicating comprises at least one of: adhering, attaching, and suturing said reinforcement with said heart.

130. The method of claim 124, wherein said configuring comprises:
communicating said reinforcement to said heart to form an anisotropic reinforcement with the heart.

131. The method of claim 130, wherein said communicating comprises at least one of: adhering, attaching, and suturing said anisotropic reinforcement with said heart.

132. The method of claim 124, wherein said direction to reinforce the infarction is determined to he the longitudinal direction of the heart.

133. The method of claim 124, wherein said direction to reinforce the infarction is determined to be the circumferential direction of the heart.

134. The method of claim 124, wherein said direction to reinforce the infarction is determined to be a direction at least substantially aligned with the underlying muscle fiber direction of the heart and/or collagen fiber direction of said infarct region.

135. The method of claim 124, wherein said direction to reinforce the infarction is determined to be a direction at least substantially transverse with the underlying muscle fiber direction of the heart and/or collagen fiber direction of said infarct region.

136. The method of claim 124, further comprising providing information to a surgeon or skilled practitioner regarding proper placement of said reinforcement.

137. The method of claim 136, wherein said information is provided at one or more of the following locations: on a surface of said reinforcement; on packaging associated with said reinforcement; on a packing label associated with said reinforcement; and/or in a set of instructions associated with said reinforcement.

138. The method of claim 136, wherein said reinforcement provides stiffness in one direction relative to other directions of said reinforcement, and wherein said information instructs the surgeon or skilled practitioner to align said one direction of said reinforcement around the circumference of an incision, opening, or defect undergoing repair.

139. The method of claim 136, wherein said reinforcement provides stiffness in one direction relative to other directions of said reinforcement, and wherein said information instructs the surgeon or skilled practitioner to align said one direction of said reinforcement around the circumference of the heart.

140. The method of claim 136, wherein said reinforcement provides stiffness in one direction relative to other directions of said reinforcement, and wherein said information instructs the surgeon or skilled practitioner to align said one direction of said reinforcement in the longitudinal direction of an incision, opening, or defect undergoing repair.

141. The method of claim 136, wherein said reinforcement provides stiffness in one direction relative to other directions of said reinforcement, and wherein said information instructs the surgeon or skilled practitioner to align said one direction of said reinforcement in the longitudinal direction of the heart.

142. A method of reinforcing a heart possessing an infarction, whereby said reinforcing creates a reinforcement to provide stiffness in one direction relative to other directions of said reinforcement, to preferentially reinforce one direction of the infarct region of the heart wall.

143. The method of claim 142, wherein said preferential reinforcement provides said stiffness in at least one direction of said reinforcement that is at least substantially aligned with the underlying muscle fiber direction of the heart and/or collagen fiber direction of said infarct region.

144. The reinforcement of claim 142, wherein said preferential reinforcement provides said stiffness in at least one direction of said reinforcement that is at least substantially transverse with the underlying muscle fiber direction of the heart and/or collagen fiber direction of said infarct region.

145. The method of claim 142, wherein said reinforcing improves heart function.

146. (canceled)
